(19) United States
(12) Patent Application Publication
(10) Pub. No.: US 2005/0266079 A1
(43) Pub. Date: Dec. 1, 2005

(54) Process for making aqueous coated beadlets

(75) Inventors: Anand Achanta, Parsippany, NJ (US); Prasad Adusumilli, Warren, NJ (US); Ganesh Deshpande, Mahwah, NJ (US); Stanley J. Lech, Rockaway, NJ (US); Philip Oths, Morris Plains, NJ (US); Arthur Vinen, Florham Park, NJ (US); Brendan Walsh, Blairstown, NJ (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/190,093

(22) Filed: Jul. 26, 2005

Related U.S. Application Data
(63) Continuation of application No. 10/088,191, filed on Jul. 16, 2002.
(60) Provisional application No. 60/153,850, filed on Sep. 14, 1999.

(51) Int. Cl. A61K 9/24; A61K 9/14

(52) U.S. Cl. 424/471

(57) ABSTRACT

The present invention is directed to application of novel process conditions for aqueous coating techniques of water soluble active agents, and its application to production of sustained release beadlets of said agents. The improvement lies in the determination and use of the glass transition point for the water swellable polymer used to produce the sustained release effect, and control of the moisture content of the air by dew point.

### BACKGROUND OF THE INVENTION

Traditional Spansule technology developed in the 1950's and still used today, utilizes a sugar pellet which is first coated with a drug/s substance followed by application of a dissolution “retarding” substance such as wax. These waxes provide the prolonged and slow release of the drug substance from the pellet. Hence, the pellet exhibits a controlled release profile over time.

The active material/drug and “retarding” wax are dissolved separately in a volatile organic solvent and applied or “layered” onto the sugar pellet in a multiple step process. In many instances, the drug/solvent and wax/solvent liquids are applied in an alternating fashion once the previous layer is dry. Drying occurs rapidly since the solvents used in the process are highly volatile and “flash” evaporated from the pellet. The process is typically carried out in rotating coating pans.

There are numerous process and safety concerns with use of this technology as summarized below.

Purchase of organic solvents for manufacturing/processing has been banned in developed countries (Montreal Agreement of 1987) due to their depleting effects on the Ozone layer.

Organic solvents are toxic, flammable and are a potential health hazard to workers.

Waxes used to retard the dissolution are naturally occurring, hence a large variation in their retarding properties upon use can occur.

The rotating pans used to process the pellets allow for operator intervention. Therefore the dissolution character of the pellet can vary from process operator to process operator. Thus, this process is deemed “difficult or un-validatable” by current Good Manufacturing Process (cGMP) practices required by regulatory agencies.

Due to the highly variable nature of these wax “retarded” pellets, numerous populations of pellets are blended together to achieve the desired final drug dissolution profile. It is not uncommon to combine 8 to 12 populations of pellets to achieve the desired dissolution effects.

Dosing of the pellets into the gelatin capsule is achieved by a single-head capsule filler that fills the capsule (by a predetermined weight) in one massive fill. Since there can be as many as 12 different lots of pellets, the relative standard variation for any one pellet could be high. These variations could potentially change the rate of release of the drug from capsule to capsule.

Capsule products may contain multiple drugs on individual beadlets and hence are more complex to (pharmacokinetically) model.

The “retarding” waxes used to control the drug release from the pellets are susceptible to rapid and adverse degradation in the gastrointestinal tract in the presence of low pH and bile salts. This situation is exaggerated with the ingestion of food. Hence, the drug release characteristics and in-vivo effect can be drastically altered (FIG. 1).

Drug release from the pellet is caused by erosion of the pellet as it moves through the gastrointestinal tract (GI). The erosion release mechanism is dated. Generally in-vivo performance of this technology is more difficult to mathematically predict and control versus newer release mechanisms such as diffusion which is used for the current invention.

Therefore, it has become necessary to utilize newer technology, which will retain the beadlets of a traditional capsule or spansule product(s), yet use an environmentally friendly, water based polymer technology, instead of organic solvents and wax. The present invention is directed towards this end.

### BRIEF DESCRIPTION OF THE DRAWINGS

FIG. 1 demonstrates traditional wax-coated spansule technology with ingestion of food.

FIG. 2 demonstrates the new aqueous coating process with ingestion of food, 75/8 formulation of PPA/CPM.

FIG. 3 demonstrates In-Vitro Dissolution Results of the effect of Surelease® on the dissolution of 50% w/w drug loaded phenylpropanolamine (PPA) from beadlets having varying rates of sustained release coatings, 3 to 18%, applied in accordance with Examples 1 to 3 herein.

FIG. 4 demonstrates In-Vitro Dissolution Results of the effect of Surelease® on the dissolution of 10% w/w chlorpheneramine maleate (CPM) from beadlets having varying rates of sustained release coatings, 4 to 18%, applied in accordance with Examples 4 to 6 herein.

FIG. 5 demonstrates In-Vitro Dissolution of Sustained Release PPA (50 mg) from 50/4 formulation beadlets having a 9% Surelease coating, in 0.1N HCl media.

FIG. 6 demonstrates In-Vitro Dissolution of Sustained Release CPM (4 mg) from 50/4 formulation beadlets having a 6.5% Surelease coating.

FIG. 7 demonstrates the effect of pH of the dissolution media, containing a phosphate buffer, on Sustained Release PPA (50 mg) from 50/4 formulation beadlets having a 9% Surelease coating.

FIG. 8 demonstrates the effect of pH of the dissolution media, containing a phosphate buffer, on Sustained Release CPM release (4 mg) from 50/4 formulation beadlets having a 6.5% Surelease coating.

FIG. 9 demonstrates CPM in vivo blood profiles following a single dose of the 50/4 formulation containing both IR/SR PPA and IR/SR CPM (1:1:1:1), or where indicated for PPA as 0.3:7 (IR:SR) in fed and fasted individuals.

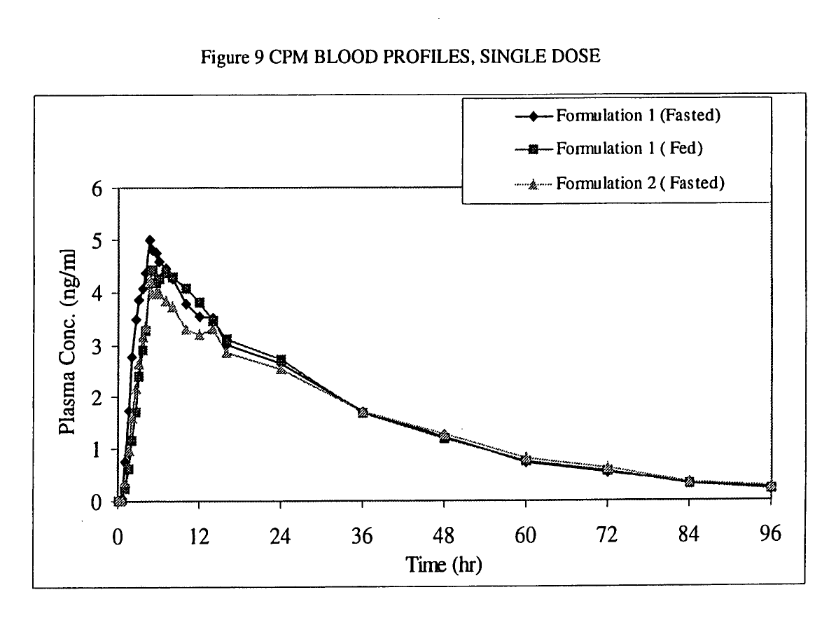

FIG. 10 demonstrates PPA In Vivo blood profiles following a single dose of the 50/4 formulation containing both IR/SR PPA and IR/SR CPM (1:1:1:1) or where indicated for PPA as 0.3:7 (IR:SR) in fed and fasted individuals.

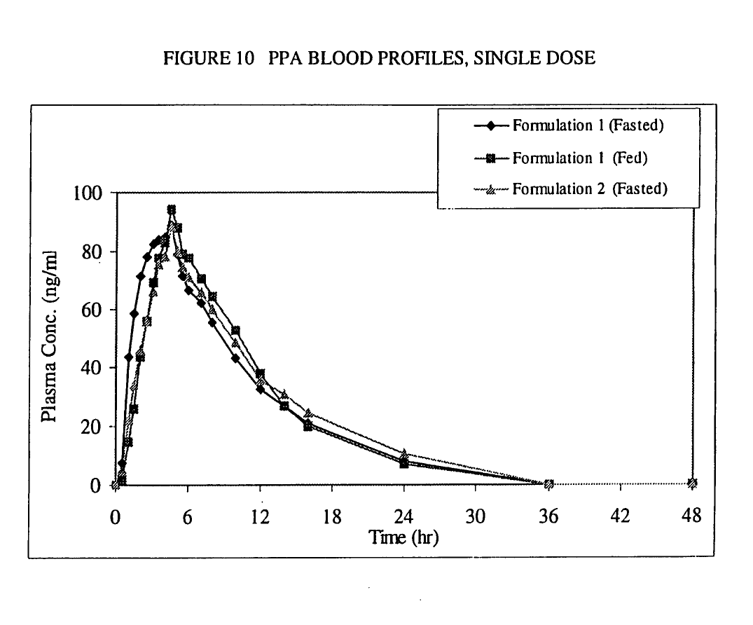

FIG. 11 CPM In Vivo Blood Levels, Single Dose 75/8 Formulation (IR:SR CPM is 1:1, 4 mg IR CPM:4 mg CPM SR). The IR Comparitor product is Chlortrimeton® 4 mg tablets, dosed at time 0, and 6 hours.

FIG. 12 demonstrate PPA In Vivo Blood Levels following a Single Dose of the 75/8 Formulation (1:2 ratio IR:SR PPA, 25 mg IR:50 mg SR). IR Comparitor Product is a 25 mg solution of PPA, given at time 0, 4, and 8 hours.

FIG. 13 demonstrates the new aqueous coating process with ingestion of food for the 75/8 mg PPA/SR formulation.

FIG. 14 demonstrates the PSE dissolution Profiles of Formulations with Different levels of Surelease Coatings.

FIG. 15 demonstrates the Effect of Media on Dissolution Rates of 10% PSE formulation and Sudafed 12 Hour Formulation.

FIG. 16 demonstrates the in vivo release rates of three PSE formulations, 6% SR, 10% SR and 14% SR, as well as the SR Comparitor, Sudafed 12 Hour caplets and IR Comparitor, Sudafed Immediate Release 30 mg tablets (6 mg dosed at 6 hours apart), using medium values.

FIG. 17 demonstrates particle size distribution curve of dextromethorphan HBr powder.

FIG. 18 demonstrates particle size distribution curve of micronized dextromethorphan HBr that is 90% less than 5 microns.

FIG. 19 demonstrate in vitro dissolution profile of Dextromethorphan HBr (DXM) pellets containing varying amounts of a sustainted release coating, 0%, a 5% and a 7%, applied in accordance with Examples 10 and 11 herein.

FIG. 20 demonstrates the in-vivo release rates of IR DXM (listed as DLSC), 5% and 7% SR DXM pellets along with the IR Comparitor, Robitussin Dry Cough syrup. This figure provides for concentration (Cp=Concentration of plasma) of Dextromethorphan in the plasma.

FIG. 21 demonstrates the in-vivo release rates of IR DXM (listed as DLSC), 5% and 7% SR DXM pellets along with the IR Comparitor, Robitussin Dry Cough syrup. This figure provides for concentration of Free Dextromethorphan in the plasma.

FIG. 22 demonstrates the in-vivo release rates of IR DXM (listed as DLSC), 5% and 7% SR DXM pellets along with the IR Comparitor, Robitussin Dry Cough syrup. This figure provides for concentration of Total Dextromethorphan in the plasma.

### SUMMARY OF THE INVENTION

The present invention is directed towards sustained release beadlets of chlorpheneramine maleate, phenylpropanolamine, pseudoephedrine and dextromethorphan having specific AUC values, Cₘₐₓ, and Tₘₐₓ values as described in the figures.

More specifically, the present invention is directed towards a product comprising a sustained release (SR) phase of PPA beadlets coated with about 9 to about 24% (weight gain) of a pseudolatex water swellable polymer dispersion.

Another embodiment is the SR product of PPA which further comprises an immediate release phase of PPA beadlets.

The immediate release phase of PPA beadlets are coated with about 0.5 to about 8% (weight gain) of a pseudolatex water swellable polymer dispersion.

Another embodiment of the present invention is a product comprising a sustained release (SR) phase of CPM beadlets coated with about 5 to about 18% (weight gain) of a pseudolatex water swellable polymer dispersion.

Another embodiment is an SR product of CPM beadlets which further comprises an immediate release phase of CPM beadlets.

The immediate release phase of CPM beadlets are coated with about 0.5 to less than 5% (weight gain) of a pseudolatex water swellable polymer dispersion.

Another aspect of the present invention is a combination product which contains a ratio of immediate release beadlets to sustained release beadlets of both chlorpheneramine maleate and phenylpropanolamine.

Another aspect of the present invention is directed towards a product comprising a sustained release (SR) phase of PSE beadlets coated with about 3 to about 20% (weight gain) of a pseudolatex water swellable polymer dispersion.

Another embodiment is an SR product of PSE beadlets which further comprises an immediate release phase of PSE beadlets.

Another aspect of the present invention is a combination product which contains a ratio of immediate release beadlets to sustained release beadlets of both chlorpheneramine maleate and pseudoephedrine.

Another aspect of the present invention is a combination product which contains a ratio of immediate release beadlets to sustained release beadlets of chlorpheneramine maleate, pseudoephedrine, and phenylpropanolamine.

Another aspect of the present invention is directed towards a product comprising a sustained release (SR) phase of Dextromethorphan HBr (DXM) beadlets coated with about 0.5 to about 15% (weight gain) of a pseudolatex water swellable polymer dispersion.

Another aspect of the present invention is the SR product of DXM beadlets which further comprises an immediate release phase of Dextromethorphan (DXM) beadlets.

Another aspect of the present invention is the ratio of IR beadlets to SR bead lets of Dextromethorphan.

Another apsect of the present invention is immediate release phase beadlets of Dextromethorphan HBr, which beadlets may be contained in a hard or soft gelatin capsule or as a unit dose sachet.

Another aspect of the present invention is a combination product which contains a ratio of immediate release beadlets to sustained release beadlets of DXM in with chlorpheneramine maleate, pseudoephedrine and/or phenylpropanolamine in varying ratios of each other.

Another aspect of the present invention relates to the use of micronized dextromethophan HBr for making IR and SR beadlets.

Yet another aspect of the present invention relates to novel process conditions for aqueous coating techniques which is:

An aqueous coating process for the manufacture of sustained release beadlets of a water soluble active agent coated with a water swellable polymer as the sustained releasing agent which process comprises
a) applying to a drug loaded sphere a seal coat of a protective polymer;
b) applying to the sphere of step a) a coating of an aqueous water swellable polymeric dispersion; wherein the aqueous water swellable polymeric dispersion of step b) is a pseudolatex ethyl cellulose dispersion having a glass transition point of about 38 to 41° C.; and which process for applying said dispersion utilizes atmospheric conditions exhibiting a dew point of <9+/−3° C.

### DETAILED DESCRIPTION OF THE INVENTION

The present invention is directed towards utilization of a new dissolution “retarding” polymer, for commercial scale production, which may be employed to provide a slow and steady release of a drug substance from pellets over an extended time period, such as a 12 hour period. For simplicity of description, and for the manufacturing and pharmacokinetic modeling necessary to achieve the desired end product, the active ingredients are preferably loaded onto separate pellets.

The term “active ingredients” as used herein, is meant to include any pharmaceutically acceptable agent having medicinal properties, such as the over-the-counter medications phenylpropanolamine hydrochloride (PPA), pseudoephedrine hydrochloride (PSE), and other amines, chlorpheniramine maleate (CPM), and other antihistamines, diphenhydramine, dextromethorphan (DXM) and its salts, loratadine (Claritin®), descarboethoxyloratadine (DCL), fexofenadine (Allegra®), and cetirizine hydrochloride (Zyrtec®), guaifenesin, acetaminophen, aspirin, ascorbic acid, cimetidine, clemastine and its salts, codeine phosphate, dextroamphetamine and its salts, dexbromopheniramine and its salts, dimenhydrinate, docusate sodium, doxylamine succinate, ephedrine salts, non-steriodal inflammatory agents, such as ibuprofen and its salts, ketoprofen and its salts, naproxen, sodium naproxen, other salts, meclizine and it salts, nicotine and it salts, nizatidine, phenylephrine and its salts, pyrilamine and its salts, salicylamide, triprolidine and its salts, etc.

Water solubility of the active agent is defined by the United States Pharmacoepia. Therefore, active agents which meet the criteria of very soluble, freely soluble, soluble and sparingly soluble as defined therein are encompassed this invention.

For purposes herein the term “beadlets” and “pellets” are used interchangeably.

It is recognized that the more water soluble the active ingredient is the better the In-vitro/InVivo Correlation (IV/IVC) will be. IV/IVC is a form of pharmacokinetic modeling described further below which was conducted on pellets produced with the process described herein. It has subsequently been determined that use of this process provides for a highly predictable outcome.

The present invention is a novel use of advanced technology as applied to over-the-counter medications, although prescription medicaments may also be used. Primarily, this new technology includes the use of 1) a Wurster Fluid Bed processor or equivalent, to ensure a more precise application of the active to each pellet, 2) drug delivery by a diffusion mechanism which uses a specialized polymer to control the medicine release with greater reproducibility, 3) separate immediate release (IR)/sustained release (SR) beads for each medicine to allow for an immediate and sustained release of drug; and 4) a multi-head capsule filler to ensure that the proper mixture of medicines with the correct ratios of IR to SR pellets is achieved in each capsule.

This technology has not previously been used within the OTC arena, and in particular for large scale medicaments, such as cold preparations. In many instances, this technology is used whereby the final product does not produce drug delivery by a diffusion mechanism but instead by an erosion process, or a combination thereof. The parameters described herein enable the skilled artisan to produce a product in which the diffusion process is directly related to the thickness of the coating polymer. No additional talc, etc. is necessary to correct for the tackiness or other properties of the polymers used in the coating process.

There are several critical elements in the production process for the OTC medicaments that mirror the production processes used for several prescription drugs such as:
1) Encapsulating pellets in a two piece hard gelatin capsule;
2) Use of pellets coated with a special water swellable polymer (such as ethylcellulose or its equivalent) to control the medicament release mechanism, allowing for sustained release of the drug substance; and
3) Use of a Wurster Fluid bed processor or equivalent to apply the special polymer coating.

While Applicants are not privy to the manufacturing details of other products, a review of publicly available information suggests that many prescription products may use these critical technological elements including, Kadian (analgesic) manufactured by Faulding, Micro-K Extendcaps (Potassium replacement) manufactured by A.H. Robins, Dilatrate-SR (cardiac, vasodilator) manufactured by Schwarz Pharma, Theo-24 (a bronchodilator) manufactured by UCB Pharma. All of these products feature pellets encapsulated in a two piece hard gelatin capsule. Based on ingredient statements, it appears that water swellable polymers are used. In turn, the application of this type of polymer typically requires a Wurster fluid bed processor or a unit of similar design. It is therefore highly likely that all three critical parameters are used in these three products.

This type of technology does not appear to have been used for any OTC medicines, based on a review of all products contained within the 1998 Physicians Desk Reference For Non-Prescription Drugs, for instance. An essential element of this technology is the use of sustained release pellets delivered within a two piece hard gelatin capsule. Within the 1998 PDR For Non Rx Drugs, there are 5 OTC, non-herbal capsule products that use a two piece hard gelatin capsule. They are: Basaljel ( Wyeth-Ayerst); Benadryl Allergy (Warner-Lambert); Contac Capsules (SmithKline Beecham); Sleepinal (Thompson); and Teldrin (Hogil Pharmaceutical).

Of these products, Basaljel, Benadryl Allergy and Sleepinal are immediate release products and are conventional powder filled capsules. These products do not contain sustained release (SR) pellets, and therefore do fit within the use of this process for SR technology.

This leaves Contac Capsules and Teldrin as the only sustained release OTC medicine products in two piece hard gelatin capsules. The technology used for the “old” Contac Capsule employed organic solvents and waxes to achieve its sustained release effect. Teldrin, a sustained release pellet, uses traditional spansule technology, i.e. a pharmaceutical glaze, commonly known as Lacquer, to retard the drug release and provide the sustained release mechanism. Both wax and Lacquer layering is traditionally carried out in rotating pans using organic solvents. Thus, no OTC medicine product applies all the critical elements of the present invention.

A beadlet using the old wax technology process is shown below:

[image]

Thus, the present invention is directed towards production of a new formulation which is quite different from previous OTC formulations, such as Contac Capsules, due to major differences in the type of beads used (different IR/SR beads for each active ingredient), differences in coating techniques (the new capsules use the patented Wurster coating process and water swellable polymer versus the pan-sprayed wax/organic solvent coating of the previous capsule), and improved batch-to batch variability.

As noted above, the prior art capsules are/were manufactured using a wax/organic solvent coating technique which does not provide consistent release profiles between similar lots of pellets. This is due to the fact that wax is a natural product and subject to a high degree of variability between lots. The present process overcomes this difficulties by utilizing a water swellable polymer, preferably ethylcellulose. Use of an ethylcellulose polymer dispersion provides for a highly reproducible product, with low lot to lot variation.

A suitable aqueous ethyl cellulose latex dispersion for use herein is Surelease®, Colorcon, PA. Alternative ethyl cellulose dispersions are available from other suppliers.

The mechanism of release for the old Contac capsule was based upon the erosion of the wax matrix in the gastrointestinal (GI) tract. This type of release mechanism is difficult to mathematically model. The new formulation's release mechanism is based on diffusion and follows standard Fickian Diffusion. This allows for easy mathematical modeling of the release of the medicine in-vivo. As evidence of the greater control diffusion release has over erosion, in-vitro tests of the new formulation provide for reliable measurements of percent release at more regular intervals. This would not have been feasible with the older erosion mechanism as the release was not as reliable at every time point.

The pellet layering/coating process of present invention is carried out in fluid bed coater. The fluid bed suspends the pellets in a continuous stream of air that passes the beads through alternating stages of coating (layering) and drying. The process consists of spraying a predetermined amount of drug onto a sugar pellet followed by a protective polymer coat, which is termed the “seal coat”. Alternatively, a spherionized pellet of the drug/active agent may be used instead of a sugar sphere loaded with drug. The spherionized pellet is also coated with a protective polymer. Later, the seal coated beads are coated with an aqueous polymeric dispersion (also termed as sustained release or functional coat) which regulates the drug release from the beads. Preferably, an aqueous ethyl cellulose latex dispersion is used as the functional, or sustained release, polymeric coat. Finally, a color coat is applied to achieve pharmaceutical elegance and consumer appeal. At this stage, beads are generally termed “sustained release” beads (SR beads). Although, for purposes herein SR beads need not include the “top coat” or “colour” coat to be referred to as SR beads.

Depending upon the solubility of the active ingredient, a small amount of the functional coating may be added for purposes of stability and to insure that the “immediate release” of the agent corresponds to the generally accepted idea that IR release occurs within about a 45 minute time period and that the product consistently releases at this rate. Therefore, the term immediate release as used herein may also include a slightly delayed response so that the drug is fully released w/in the generally accepted parameters of an immediate release dosage form. For instance, PPA is highly soluble and hence the IR beadlets require a small amount of functional coating to produce a bioequivalent product to the art recognized IR tablets on the market. CPM performs similarly. However, in contrast both PSE and the DXM IR pellets do not require functional coating in the IR phase.

The type of sphere onto which the active ingredient is loaded is well with the skilled artisan's choice. Generally, the spheres are sugar spheres, such as sucrose, however microcrystalline cellulose, such as Avicel®, is also a suitable alternative. If the osmotic nature of the sphere is increased, there will be an increase in the diffusion rate of the active moiety as well. All of which parameters need to be taken into account with the thickness of the functional sustained release coating.

The protective polymer coating used as a “seal coat” is well recognized by the skilled artisan who would readily determine a suitable barrier coating agent. Preferred barrier coats for use herein with the pseudolatex ethyl cellulose dispersion coating is one which contains polyvinyl alcohol, such as Opadry AMB®, or hydroxypropylmethylcellullose (HPMC), hydroxypropylcellulose (HPC), acrylic polymers such as Carbopol, or an enteric coating which is water dispersible/soluble and pH sensitive, such as several of the Eudragit® coatings.

[image]

To provide initial and rapid (immediate) availability of the drug, some drug layered and seal coated beads are coated with a substantially reduced functional (sustained release) coat and then color coated. These beads are termed “immediate release” (IR) beads. For purposes herein IR beads need not include the “top coat” or “colour” coat to be referred to as IR beads. As necessary, one may use a predetermined composition of IR and SR beads to achieve the desired pharmacological effect. To do so, the predetermined amounts of each bead are filled into a two piece hard gelatin capsule using suitable, well and well known high speed, multi-head capsule filling machines. A soft gelatin capsule may also be used. The ratio of IR to SR beads in a mixture may be varied in order to obtain the desired blood levels and comply with appropriate regulatory requirements of any particular country.

Benefits of the instant process include use of water as a solvent/carrier, which is safe to humans and environmentally friendly. The process also uses ethylcellulose, not waxes (as used in the prior art) to provide the sustained release effects. Ethylcellulose modulates drug release in a highly consistent and predictable manner under various gastrointestinal/biological and simulated conditions. This illustrates that drug release for the new technology is virtually the same across multiple conditions, unlike traditional wax coated pellets. Other benefits include no toxic emissions or aging time of the product, improved economic manufacturing conditions.

For purposes herein Opadry® AMB Pink, is a product produced by Colorcon, West Point, Pa., having a composition of Polyvinyl alcohol, partially hydrolyzed (USP, JPS); Talc, Alumina Hydrate, titanium dioxide, carmine, lecithin, and xanthan gum.

For purposes herein Opadry® Pink, is a product produced by Colorcon, West Point, Pa., having a composition of HPMC 2910/hypromellose 3 cp, HPMC 2910/hypromellose 6 cp, titanium dioxide, macrogol/PEG 400, and carmine.

For purposes herein Opadry® yellow, is a product produced by Colorcon, West Point, Pa., having a composition of HPMC 2910/hypromellose 3 cp, HPMC 2910/hypromellose 6 cp, titanium dioxide, macrogol/PEG 400, iron oxide yellow, and polysorbate 80.

For purposes herein Surelease® clear, is an aqueous pseudolatex of ethylcellulose, produced by Colorcon, West Point, Pa., having a composition of purified water, ethylcellulose, ammonium hydroxide, medium chain triglycerides, and oleic acid.

For purposes herein, AquaCoat® is a product produced by FMC Corporation, Philadelphia, Pa. Both Aquacoat and Surelease are pseudolatex ethylcellulose dispersions.

For purposes herein, Sugar spheres, NF are spherical particles used as a substrate onto which the drug or active agent is loaded. Sugar spheres, NF contain mostly sugar (62.5-91.5%), with the remainder consisting of starch. For purposes herein, Methocel E5, Premium (hydroxypropylmethyl cellulose, USP/NF) is a Dow chemical grade product for a hydroxypropyl substitution specification of 7-12% and a methoxyl substitution specification of 28-30%. These substitution specifications meet the requirements for hydroxypropyl-methylcellulose USP substitution #2910 (1). Where appropriate, Methocel E5, Premium, is used in the pellet formulations of the present invention with drug solution, to help the drug adhere to the sugar sphere surface and, used to “seal” the Drug Loaded Sugar Core (DLSC) pellets, after the drug has been applied.

It is recognized that the skilled artisan may chose to use other pharmacologically inactive spherical seed cores for use herein, and it is contemplated that all such spheres are within the context of this invention. One suitable alternative is a sphere composed primarily of microcrystalline cellulose.

For purposes of illustration of the process, a capsule containing 4 populations of pellets has been prepared and is described in the Examples below. Each pellet fraction is dosed separately and filling is accomplished by a high speed multi-head capsule filler, such as an MG2 Futura. Using the active ingredients, phenylpropanolamine hydrochloride (PPA) and chlorpheniramine maleate (CPM) the pellets are given the designations: CPM IR for chlorpheniramine immediate release: CPM SR for chlorpheniramine sustained release; PPA IR and PPA SR for phenylpropanolamine immediate and sustained release respectively. Similarly, pellets of IR PSE, SR PSE, IR DXM and SR DXM represent immediate release pseudoephedrine, sustained release pseudoephedrine, immediate release dextromethorphan and sustained release dextromethorphan respectively.

The fact that the process used herein is highly reproducible allows it to meet all global regulatory standards in terms of process validation and cGMP's. The process utilizes a one-polymer system to modulate drug release. This system has distinct advantages over the wax “retarded” systems developed in the 1950's as this process provides for only one drug per pellet, which makes designing in-vivo release profile for combination products more predictable and controllable than the previous two drugs per pellet.

Many different formulation prototypes were developed using different drug loading, seal coating and functional coating membranes. These formulations were analyzed for their drug content (assay), dissolution profiles, and stability at ambient and accelerated conditions.

Release from the beads coated with ethylcellulose polymer follows standard Fickian Diffusion and is highly predictable and is based on the following equation:
Mₜ/M_{inf}=ktⁿ

Where Mₜ/M_{inf} is the fraction of drug released at time t, k is the proportionality constant; n is the exponent characteristic of the mode of transport. In most of the cases, release from the spheres is proportional to the square root of time, i.e. n=0.5.

During experiments it was observed that the thickness of functional coat (Surelease®, 25% w/w ethyl cellulose) controls the rate of drug release. To systematically investigate this issue, beads were coated with different amounts of Surelease® and studied for their drug release at ambient and accelerated conditions. In this exercise, the quantitative formula was kept constant for both PPA and CPM (as shown in Table 1 below), and only the Surelease® level was changed to study the effect of varying polymer levels.

For purposes herein “functional coat” and “sustained release coat” are unsed interchangeably.

It is recognized that the thickness of the seal coat, the thickness of the sustained release coat and the permeability of the sustained release coat will play a role in the diffusion profile of the active agent in the beadlet. Consequently, it is the process parameters as defined herein which allow the skilled artisan to achieve reproducibility of the product and stability of the product.

| PPA Beads: | | |
|---|---|---|
| Drug Loading (PPA): | 50% | weight gain |
| Seal Coat (Opadry AMB white): | 5% | weight gain |
| Functional Coat (Surelease ®): | 3 to 24% | w/w |
| Color Coat (Opadry AMB Pink): | 2% | weight gain |
| CPM Beads: | | |
| Drug Loading (CPM): | 10% | weight gain |
| Seal Coat (HPMC E-5): | 2% | weight gain |
| Functional Coat (Surelease ®): | 2 to 18% | weight gain |
| Color Coat (Opadry Yellow): | 2% | weight gain |
| PSE Beads: | | |
| Drug Loading (PSE): | 60% | weight gain |
| Seal Coat (HPMC E-5): | 2% | weight gain |
| Functional Coat (Surelease ®): | 6 to 14% | weight gain |
| Color Coat (Opadry Pink): | 2% | weight gain |
| DXM Beads: | | |
| Drug Loading (PSE): | 50% | weight gain |
| Seal Coat (HPMC E-5): | 2% | weight gain |
| Functional Coat (Surelease ®): | 6 to 10% | weight gain |
| Color Coat (Opadry Pink): | 2% | weight gain |

Shown in Table 1 below are actual examples of % functional coat used in preparation of beadlets pursuant to the procedures shown in the working examples:

TABLE 1
The percent of Surelease ® used to coat seal coated beads

Surelease ® Levels (weight gain)

| | | | | | | |
|---|---|---|---|---|---|---|
| PPA | 3% | 6% | 9% | 14% | 18% | 24% |
| CPM | 2% | 5% | 6.5% | 12% | 16% | 18% |

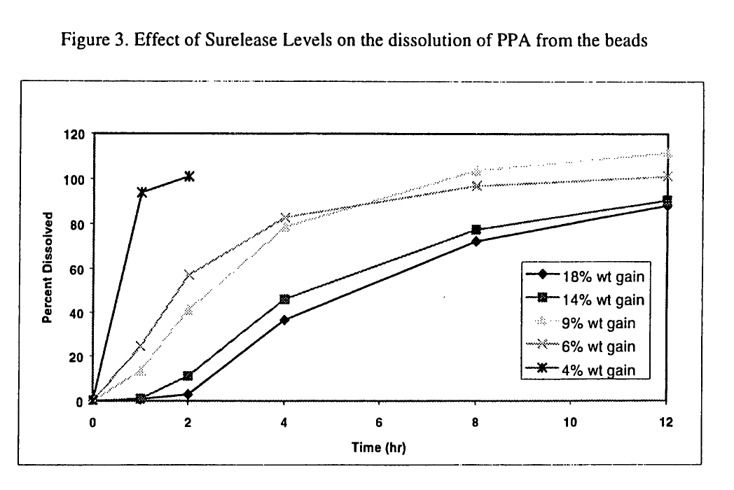

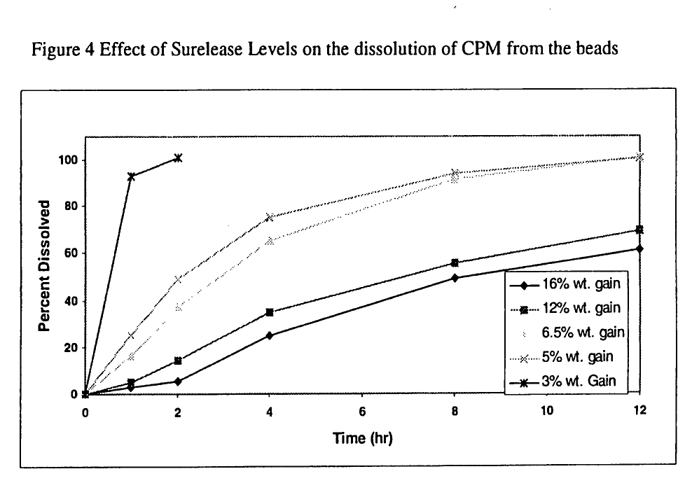

Mean dissolution values for both PPA and CPM are shown in FIGS. 3 and 4, respectively. For both PPA and CPM, a systematic increase in polymer content (Surelease®) decreases the release rate of the drug. This is also applicable to PSE, and the DXM pellets of the present invention and is likely to be consistent for all the active agents listed herein. It is obvious from the plot that if the polymer level in the formulation is known, in-vitro dissolution can be estimated with reasonable accuracy.

Based on these experiments it was determined that beads coated with 3 to 24%, preferably 9 to 18% of Surelease® for PPA and 2 to 18%, preferably 6 to 16% of Surelease® for CPM (more preferably 6.5 to 9%) show satisfactory stability up to six months under accelerated conditions.

These formulations were subjected to stability evaluation at room temperature and at accelerated conditions (40° C./75% RH). At room temperature, all beads showed good stability and release profiles compared to their initial release.

Alternative formulations were developed to evaluate the in-vivo release performance of the beads. Formulations containing beads with 9% Surelease® for PPA and 6.5% Surelease® for CPM were selected with the PPA beadlets containing 50 mg of PPA and the CPM beadlets containing 4 mg of CPM. It is recognized that the amount of active ingredient may vary, such as beadlets containing 75 mg PPA and 8 mg CPM. It is also recognized that the amount of functional coat of Surelease may also vary. However, two preferred formulations are shown below:

Formula (1): PPA SR: 50% PPA Loaded on a Sphere/5% Opadry (Seal Coat)/9% Surelease® (Functional Coat)/2% Opadry (Top Coat)

CPM SR: 10% CPM loaded on a sphere/2% HPMC (seal coat)/6.5% Surelease® (functional coat)/2% Opadry Yellow

Formula (2): PPA SR: 50% PPA Loaded on a Sphere/5% Opadry (Seal Coat)/12% Surelease® (Functional Coat)/2% Opadry (Top Coat)

CPM SR: 10% CPM loaded on a sphere/2% HPMC (seal coat)/8% Surelease® (functional coat)/2% Opadry Yellow

Another aspect of the invention is a preferred formulation of IR beads (alone) or in combination for and IR phase of an admixture.
PPA IR: 50% PPA loaded on a sphere/5% Opadry (seal coat)/4% Surelease® (functional coat)/2% Opadry (top coat)
CPM IR: 10% CPM loaded on a sphere/2% HPMC (seal coat)/3% Surelease® (functional coat)/2% Opadry Yellow

Another aspect of the invention is a combination of 3 or more SR beadlets in the extentended or sustained release phase/portion of the admixture, such as the following preferred formulation:
PPA SR: 50% PPA loaded on a sphere/5% Opadry (seal coat)/18% Surelease® (functional coat)/2% Opadry (top coat)
PPA SR: 50% PPA loaded on a sphere/5% Opadry (seal coat)/11 or 12% Surelease® (functional coat)/2% Opadry (top coat)
CPM SR: 10% CPM loaded on a sphere/2% HPMC (seal coat)/8% Surelease® (functional coat)/2% Opadry Yellow

Suitably, the range of PPA in an IR:SR formulation is from 1:1 to about 1:6. One embodiment is a ratio of 0.3:0.7. The range of CPM in an IR:SR formulation is about 1:3, preferably about 1:1. Given the particular type of drug a ratio of IR:SR will generally be from about 1:6.

Also contemplated herein are pediatric dosage forms with pellets produced in accordance with the teachings herein. A suitable dosage amount for pediatric use for IR to SR for PPA is 6.25 to 12.5 mg IR PPA:37.5 to 44 SR PPA; and a suitable dosage amount for CPM is IR to SR for CPM is 0.5 to 3.5 mg IR CPM:2 to 4 mg SR CPM. Dextromethorphan is about 2.5 mg IR L 2.5 mg SR.

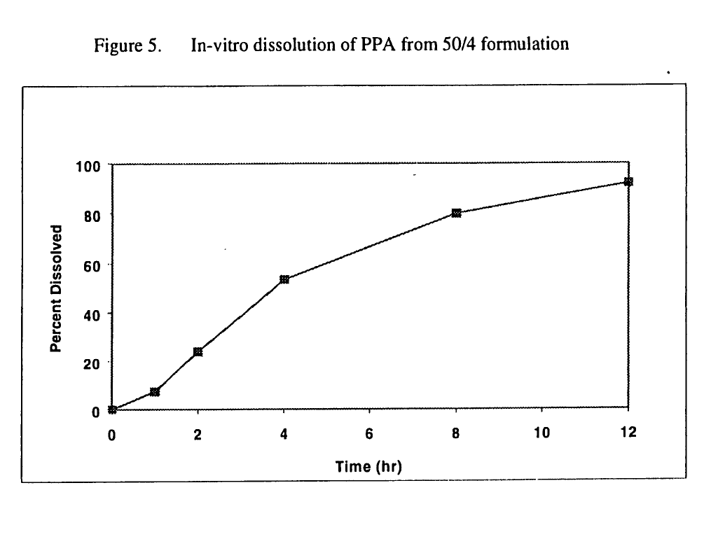

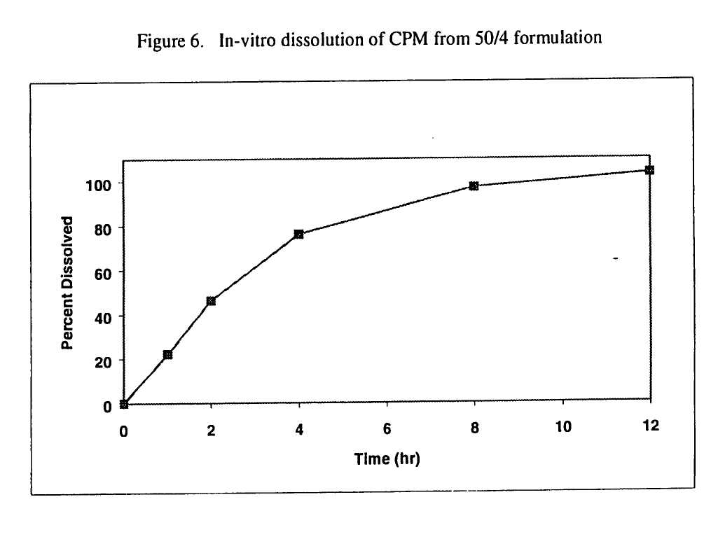

The PPA SR and CPM SR beads above were each encapsulated into a hard gelatin capsule. In order to evaluate the performance of this extended release technology, no immediate release beads were added to the capsules. The capsules were analyzed for drug content and in-vitro dissolution profile. The in-vitro dissolution profiles are shown in FIGS. 5 and 6.

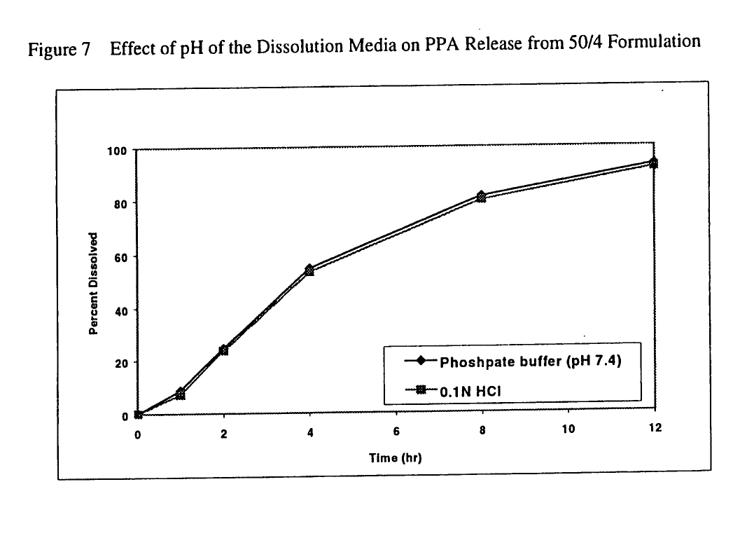

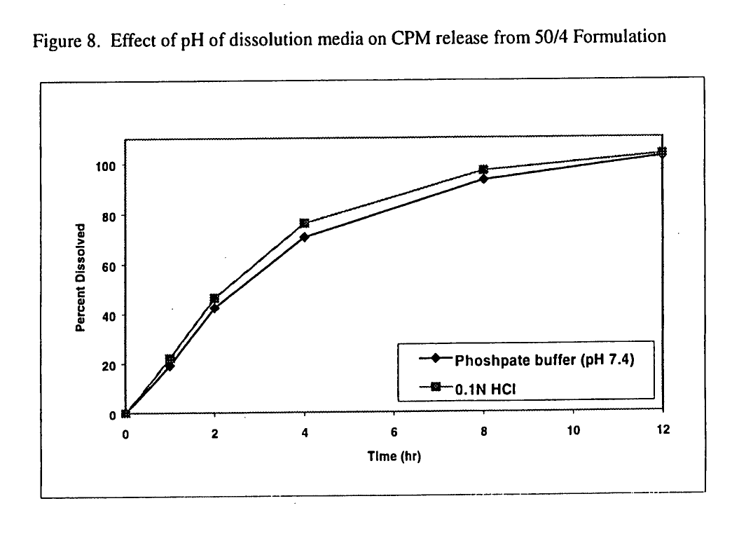

Dissolution studies of the above noted formulation were conducted in tenth normal hydrochloric acid (0.1N HCl) and in phosphate buffer of pH 7.4. Identical profiles obtained at both conditions suggest that the release is independent of the pH of the dissolution media (FIGS. 7 and 8).

Immediate release beads (IR) beads of both PPA and CPM have been produced as shown in the working Examples (dissolution data for the IR pellets is shown in FIGS. 3 and 4).

Therefore, another aspect of the present invention is the immediate release and coated (IR) form of the active ingredients, such as CPM, PPA, PSE and DXM as produced by the process herein, as well as the sustained releases forms of CPM, PPA, PSE and DXM produced by the process herein.

The IR pellets require a seal coat as these drug loaded and sealed pellets will pick up moisture, have decreased stability, and are deliquescent, i.e. they pick up moisture and dissolve in their own liquid. The moisture they pick up comes from the environment. The gelatin capsule (hard or soft) contain 12-18% moisture and the active agents pick up moisture from the gelatin capsule. Consequently, the capsule becomes brittle over time, deforms, pinholes form in the capsule shell, the active agent has decreased stability due to these changes, and the dissolution profile of the finished product is altered. Therefore, it is important to coat the drug loaded pellets with a layer of a suitable water swellable polymeric dispersion (seal coat). This aspect will be discussed in greater detail below.

Another aspect of the present invention is the blending of the IR and SR beads of both PPA and CPM in a dosage form, as well as the blending of IR and SR beads of PSE and CPM. Suitably, the dosage form is a gelatin capsule, preferably a hard gelatin capsule. Each capsule may contain IR PPA and SR PPA, or IR CPM and SR CPM in any variation of ratio or coating thickness. Alternatively all 4 populations may be blended together: IR PPA, SR PPA, IR CPM and SR CPM in any suitable ratio, and/or coating thickness. While this is essentially a biphasic system for each active moiety, it is recognized that multi-phasic systems can be prepared which have not only immediate release of an active component if desired, but release of the active moiety over a number of time points. In a similar manner each capsule may contain IR PSE and SR PSE, or IR CPM and SR CPM in any variation of ratio or coating thickness. Alternatively all 4 populations may be blended together: IR PSE, SR PSE, IR CPM and SR CPM in any suitable ratio, and/or coating thickness. In yet another emodiment, each capsule may contain IR DXM and SR DXM with IR PSE and SR PSE, IR CPM and SR CPM, or IR PPA and SR PPA in any suitable ratio, and/or coating thickness.

For purposes herein a preferred ratio for a 12 hour time release with a two component IR/SR beadlet for each active moiety is:

IR PPA:SR PPA 1:5, preferably 1:2, or a 0.3:0.7 ratio

IR CPM:SR CPM 1:4, preferably 1:1.

In-vitro/InVivo Correlation (IV/IVC) form of pharmacokinetic modeling, has been conducted on these pellets and has determined that use of this process provides for a highly predictable outcome.

The purpose of In vitro/In vivo correlation is to model in vivo response as a function of the in vitro data and use as a predictive tool in development
In vivo response=ƒ(In vitro data)

The in vivo response is dependent upon the concentration of the drug in plasma, whereas in vitro data is determined using a USP dissolution test for the particular drug in question.

To establish the in vitro-in vivo correlation, in vivo blood concentration data upon the dosing of the drug is converted into cumulative fraction absorbed (Fa) using the Wagner-Nelson method for studying absorption pharmacokinetics. In vitro dissolution studies were carried out at varying pH conditions. When in vivo fraction absorbed is compared with the in vitro dissolution, for instance in 0.1% SLS in water and 0.1N HCl, an acceptable correction level may be observed. For the examples herein of CPM and PPA, and acceptable correlation was had.

Results of these biostudies provide a good correlation between in vitro drug release and in vivo drug absorption. Thus, the established IV/IVC may then be used to determine the desired in vitro profile that would match the observed in vivo absorption profile of a predetermined drug. To match the profile of that drug, in vivo, it is required that the dose for the active agents be a combination of immediate release and sustained release components. The IV/IVC is also used to generate an array of in vitro profiles using a predetermined type of IR and SR beads (in terms of polymer coating) and different amounts of coating on the beadlets as well as the ratio of IR and SR components.

Using this system it was found that comparing the new process technology of the examples herein to the old wax coated product of Contac that biologically, overall equivalency was not achieved.

Biologically, the formulations of PPA SR and CPM SR have AUC's, Cmax and Tmax, which are the same as (or similar to) the immediate release. In other words, a 75 mg dose of PPA is biologically equivalent to three 25 mg immediate release PPA doses taken every 4 hours.

The U.S. bioequivalence criteria is that the 90% confidence interval for the ratio of the means of the AUC₀₋₁₂ and Cₘₐₓ should lie completely within the range 0.80-1.25 for log transformed data. Canada has the same criterion as the U.S. for AUC₀₋₁₂ but Canadian guidelines require only that the ratio of means for Cₘₐₓ lie within the range 0.80-1.25 (not the confidence interval of the ratio of the means).

Therefore, it is contemplated that the scope of the invention includes the full breadth of bioequivelence of the data presented in all the figures herein and not be solely limited to acutal ratio of the means.

As demonstrated herein in the accompanying figures:

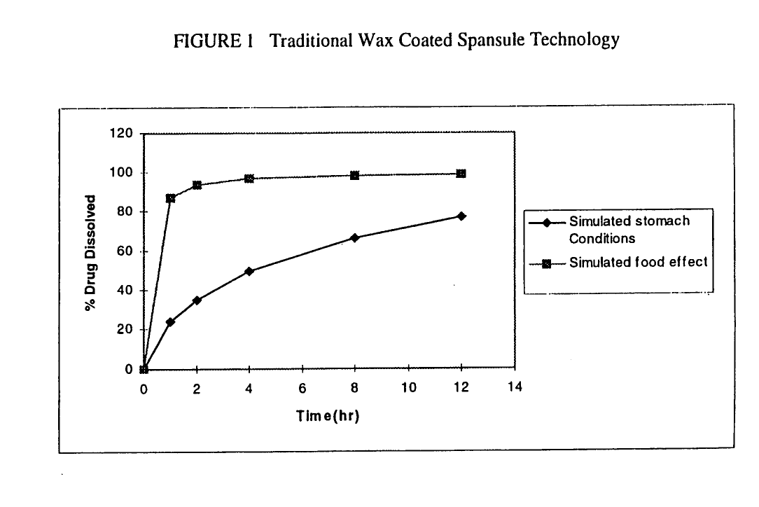

FIG. 1 is a demonstration of traditional wax coated spansule technology with stimulated food effects, with Contac 12 hour. The simulated stomach dissolution assay is performed in USP 1 apparatus and in 0.1N HCl media. The simulated food effect assay is the same as above but includes anionic surfactant to simulate emulsifying aspects of gastric juices. The simulated food effects illustrate high variability in the release of the active agents under fed conditions.

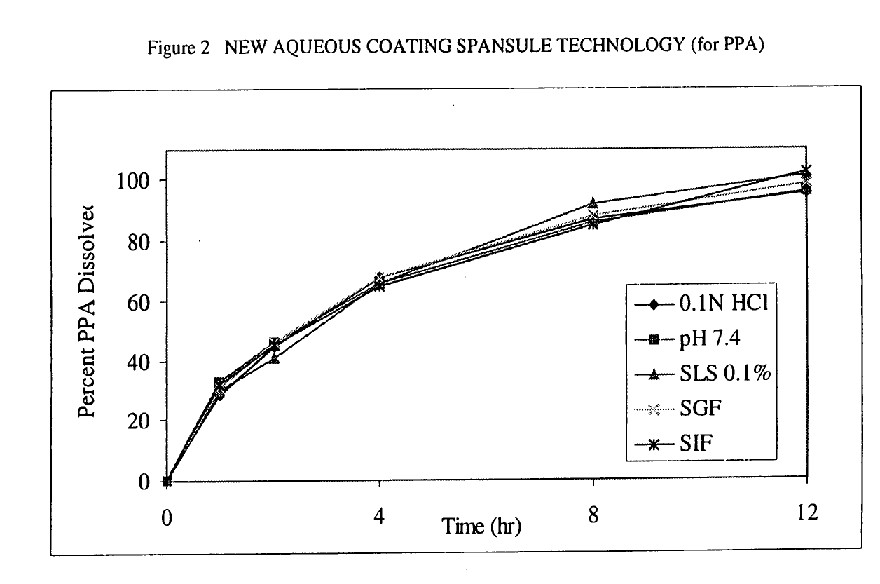

FIG. 2 is a demonstration of aqueous coated spansules of the present invention. The active agent is PPA 75/8 formulation. The simulated stomach assay is shown by the 0.1N HCl line, the simulated intestine assay performed in a USP 1 apparatus, with phosphate buffer of pH 7.4 is shown as pH 7.4 line. The simulated food effect is shown as the SLS 0.1% line. The SGF line is simulated gastric fluid, USP, and the Simulated Intestinal USP assay is shown as the SIF line. This graph illustrates minimal variability under food effects, in stomach and in intestinal fluids.

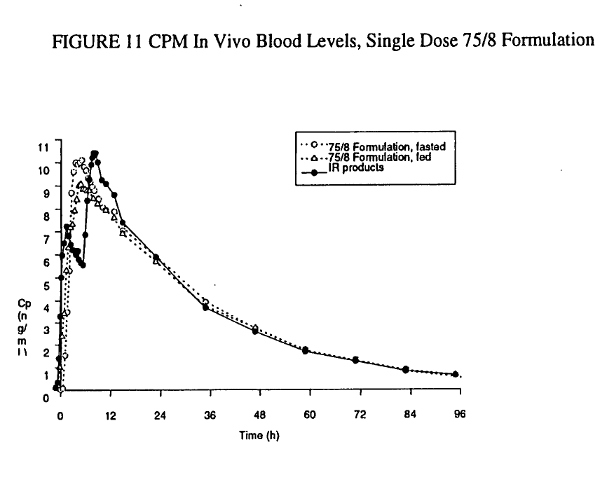

In FIG. 11, the Contac 12-Hour Fasted formula is a 1:1 ratio of IR:SR of CPM, or 4 mg IR:4 mg SR. The Comparitor product was Chlortrimeton 4 mg tablets, given at time 0 and 6 hours. The figure provides AUC, Cₘₐₓ and tₘₐₓ parameters:

For the parameter AUC_{0-∞} the comparison 75/8 formulation vs the IR Comparitor is bioequivalent (CI: 0.99-1.08).

For the parameter AUC₀₋ₜ the comparison 75/8 vs Comparitor is bioequivalent (CI: 0.98-1.08).

For the parameter Cₘₐₓ the comparison 75/8 vs Comparitor is bioequivalent (CI: 0.93-1.04).

For Contac 12-Hour® Sustained Release Capsules, the comparison 75/8 vs 75/8, (fed vs fasted), the parameters, AUC_{0-∞}, AUC₀₋ₜ and Cₘₐₓ are bioequivalent.

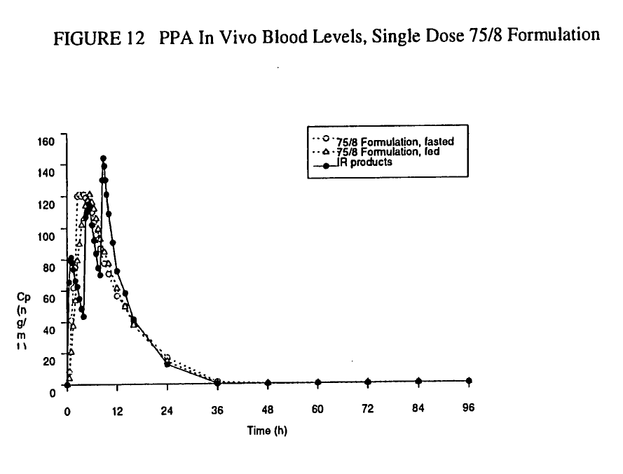

In FIG. 12, the Contac 12-Hour Fasted is a 1:1 ratio of IR:SR of CPM, or 4 mg IR:4 mg SR. The Comparitor product was a 25 mg PPA solution, given at time 0 4, and 8 hours. The figure provides AUC, Cₘₐₓ and tₘₐₓ parameters:

For the parameter AUC_{0-∞} the comparison 75/8 vs Comp is bioequivalent (CI: 0.98-1.06).

For the parameter AUC₀₋ₜ the comparison 75/8 vs Comp is bioequivalent (CI: 0.95-1.03).

For the parameter Cₘₐₓ the comparison 75/8 vs Comp is bioequivalent (CI: 0.85-0.94).

For Contac 12-Hour® Sustained Release Capsules, the comparison 75/8 vs 75/8, (fed vs fasted), the parameters, AUC_{0-∞}, AUC₀₋ₜ and Cₘₐₓ are bioequivalent.

While not shown herein, in a multidosing study using the 75/8 formulation of PPA (1:2 or 25 mg IR PPA:50 mg SR PPA), and CPM (1:1, 4 mg IR:4 mg SR), and as a Comparitor product, 4 mg Chlortrimeton tablets, 1 tablet every 6 hours, and for the IR PPA, 25 mg tablets of Propagest®, given every 4 hours, for six days.

For the parameter Area Under the Curve (AUC) the present 75/8 formulation of PPA has been found bioequivalent as compared to immediate release (log transformed 90% CI 1.00-1.04).

For the parameter Cₘₐₓ the present formulation of PPA is bioequivalent compared to immediate release (log transformed 90% CI 1.01-1.08).

For the parameter Cₘᵢₙ the present formulation of PPA is significantly smaller compared to immediate release (log transformed 95% CI 0.79-0.87).

For the parameter AUC the present formulation of CPM is bioequivalent compared to immediate release (log transformed 90% CI 0.94-1.00).

For the parameter Cₘₐₓ the present formulation of CPM is bioequivalent compared to immediate release (log transformed 90% CI 0.96-1.03).

For the parameter Cₘᵢₙ the present formulation of CPM is significantly smaller compared to immediate release (log transformed 95% CI 0.87-0.98).

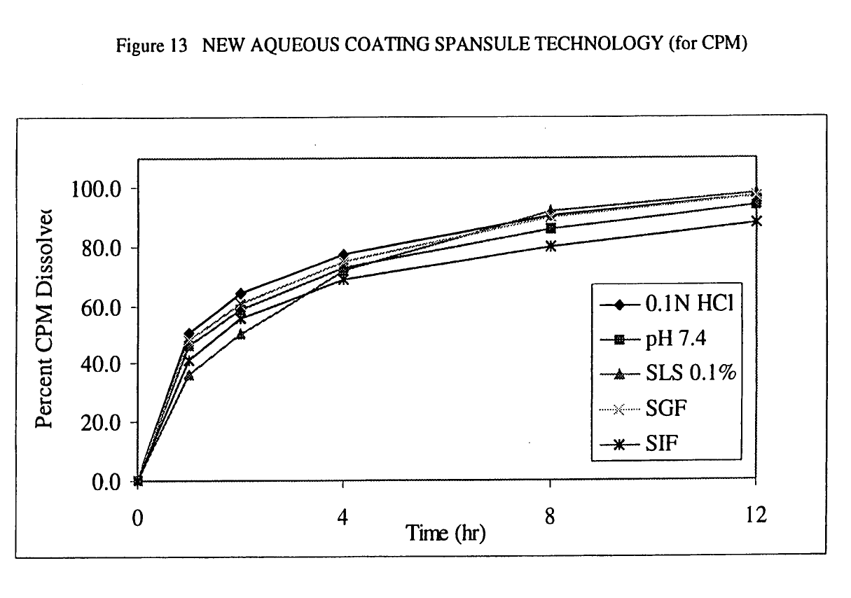

FIG. 13 is a demonstration of aqueous coated spansules of the present invention. The active agent is CPM 75/8 formulation. The simulated stomach assay is shown by the 0.1N HCl line, the simulated intestine assay performed in a USP 1 apparatus, with phosphate buffer of pH 7.4 is shown as pH 7.4 line. The simulated food effect is shown as the SLS 0.1% line. The SGF line is simulated gastric fluid, USP, and the Simulated Intestinal USP assay is shown as the SIF line. This graph illustrates minimal variability under food effects, in stomach and in intestinal fluids.

As noted above, the present invention is also directed to a pseudoephedrine HCl (PSE) immediate and sustained release capsule formulations developed by the process conditions and parameters as shown and described herein.

The in-vitro drug release profile of the new PSE pellet formulation is not affected by changes in the dissolution media pH, the presence of an anionic surfactant in the media or by the ionic strength of the media.

Similar to the manufacture of CPM and PPA, the manufacture of the PSE pellets are carried-out by first loading the PSE onto a sphere, preferably a sugar sphere, by spraying an aqueous solution of the drug with a binder. The drug-loaded pellets were then “sealed” by spraying on a layer of hydroxypropylmethylcellulose (HPMC) to which a functional coat (if desired for SR) is applied. Finally, a water-soluble topcoat is applied as an aqueous suspension to provide protection and color to both the IR and SR pellets.

As with CPM and PPA, critical processing parameters were found to include pellet bed temperatures while pellet spraying, solution/suspension spray rates and, inlet air dew point temperatures during ethylcellulose.

In a pharmacokinetic comparison, the results showed that three formulations, a “fast” formula, a “moderate” or “target” formula, and a “slow” releasing formula were bioequivalent to the Comparitor products, a Sudafed 12 Hour 120 mg caplet and to Sudafed immediate release tablets, with respect to AUC₍ₒ₋ₜ₎, AUC_{(0-inf)}, and Cₘₐₓ.

The moderate releasing capsule formulation (a 10% SR coat) overall for subjects averaged plasma drug levels vs. time profile was the most similar to the Sudafed 12 Hour caplet averaged plasma vs. time profile

Again, similar to CPM and PPA, an in vitro-in vivo correlation (IV/IVC) was established for the three pseudoephedrine HCl formulations (fast, target and, slow releasing) using the Wagner-Nelson method.

(+)-Pseudoephedrine HCl (d-pseudoephedrine HCl); (1S, 2S)-2-Methylamino-1-phenyl-1-propanol hydrochloride (CAS no. [345-78-8]), has a molecular formula of C₁₀H₁₅NO.HCl and a molecular weight of 201.69. Ephedrine and pseudoephedrine are diesteromers, the former having the erythro and the latter the threo configuration.

Pseudoephedrine HCl is a fine, white to off-white, practically odorless, crystalline or powder material. It melts between 182° and 186° C. and is very freely soluble in water, freely soluble in ethanol and, soluble in chloroform. It has an optical rotation [α]D²⁰+62° and a pKa of 9.22 (1, 2, 3).

Pseudoephedrine HCl can be found in many “over-the counter” as well as prescription cold, flu and hay fever preparations. The elimination half life of d-pseudoephedrine HCl is 5-8 hours and a number of immediate release formulations on the market are commercially available with a dosing recommendation of 60 mg every six hours. Commercial sustained release preparations of pseudoephedrine HCl typically have a recommended dosing regimen of 120 mg of pseudoephedrine HCl every 12 hours.

Therefore, a preferred formulation of this invention is a formulation designed to release 120 mg of (+)-pseudoephedrine hydrochloride from, preferably, a hard gelatin capsule, and preferably over a 12 hour time course.

An improvement seen with the new pseudoephedrine hydrochloride 120 mg formulation versus currently available formulations is the use of a single population of pellets. Eliminating multiple pellet populations greatly reduces processing time and serves to simplify the drug product manufacture. Using a single population of SR pellets also increases batch to batch drug release profile consistency due to the use of a single lot of pellets.

As discussed previously, the in-vitro drug dissolution profiles of the formulations of this invention, using baskets (USP apparatus II) can be modulated using different levels of Surelease. Increasing the level of Surelease® gives slower drug release rates. The drug release rate can also be modulated by changing the drug load, however, this approach is not preferred because drug release rate is far less sensitive to changes in the level of drug loading than to changes in the level of the Surelease barrier coat.

Suitably, the drug loading onto the sugar spheres for PSE may range from 6% to 90% weight gain. Preferably, 40 to 75% and more preferably 50 to 70, and most preferably 55 to 66%.

Seal coating for PSE can vary somewhat from about 0% to about 20%. Preferably, the seal coat is less than 10%, more preferably less than 5%, and most preferably about 2%.

Functional coating of the pseudolatex water swellable polymer dispersion for PSE may range from about 3 to 20%, preferably 6 to 14% and more preferably about 10 to 12% w/w.

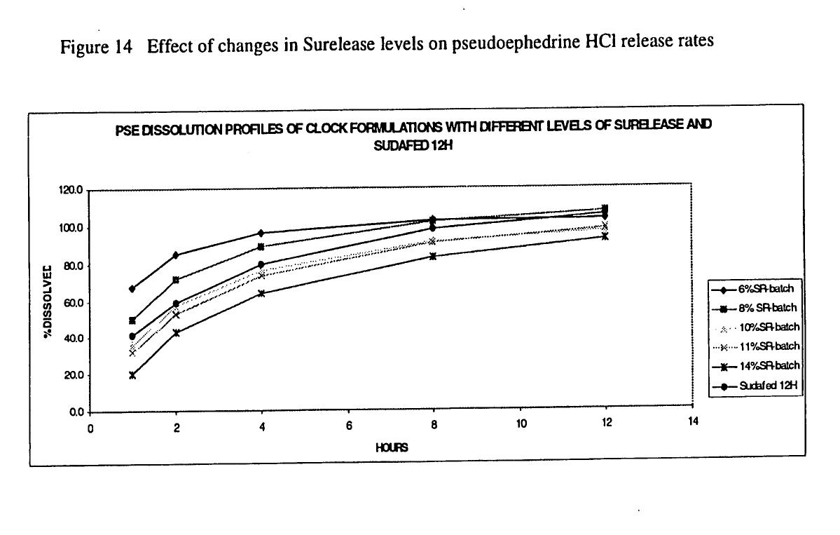

FIG. 14 demonstrate the in-vitro dissolution profiles of different Pseudoephedrine HCl 120 mg SR pellet capsule formulations coated with 6%, 8%, 10%, 11% and 14% Surelease® levels. Also shown on the plot is the dissolution of the Comparitor product, Sudafed 12 Hour Caplets, which were purchased at a local pharmacy.

The dissolution profile data shows that increasing the Surelease® level results in decreasing drug release rates. The dissolution profile for the 8% SR formulation is between the 6% and 10% profiles. Likewise, the profile for the 10% formulation lies between the profiles for the 8% and the 11% formulations. Finally, the 11% formula lies between the 10% and 14% formulations. The Sudafed® 12 Hour profile most closely matches that of the 10% SR.

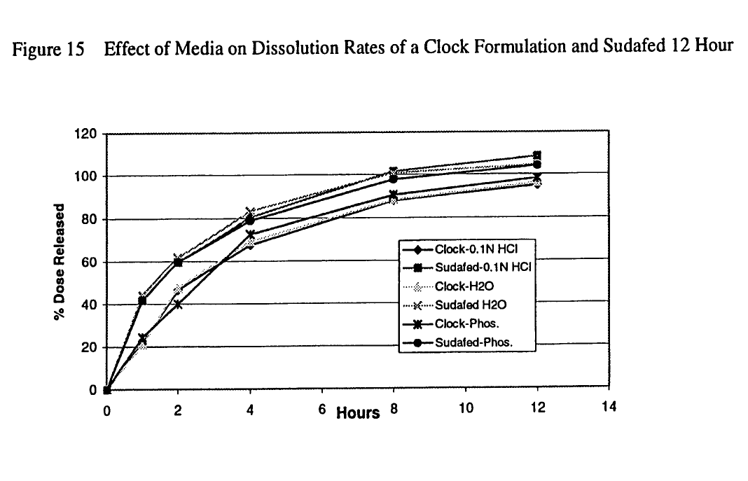

Changing the dissolution media on the release rate of, for instance, a 12% Surelease pellet formulation of PSE and for Sudafed 12 Hour (which also delivers 120 mg of PSE) demonstrated that dissolution of PSE from either formulation was not affected by changes in pH or buffer content. See FIG. 15, for effect of media on the dissolution rates.

Consequently, PSE pellets are suitably produced herein with a % functional coat of Surelease for a SR bead of 6% to 14% SR. The 6% SR is considered a “fast” releasing formulation, whereas the 14% are considered a “slow” releasing formulation. A “moderate” releasing formulation would be considered a 10 to 12% SR formulation. The in-vitro release rate of the 10% SR formula released drug approximately 25% slower and 25% faster than the 6% and 14% SR formulations, respectively.

It is recognized that combinations of sustained release to immediate release pellets of PSE may be used herein. Suitable weight ratio of immediate release PSE to sustained release PSE is at its broadest useage, 0.1:1 to 1:0.1, with approximately 8:1 to about 1:1 being prefered. In terms of mg dosage amounts this would result in a weight ratio dose of 15 mg immediate to 105 mg SR, to a 60 mg IR:60 mg SR dose.

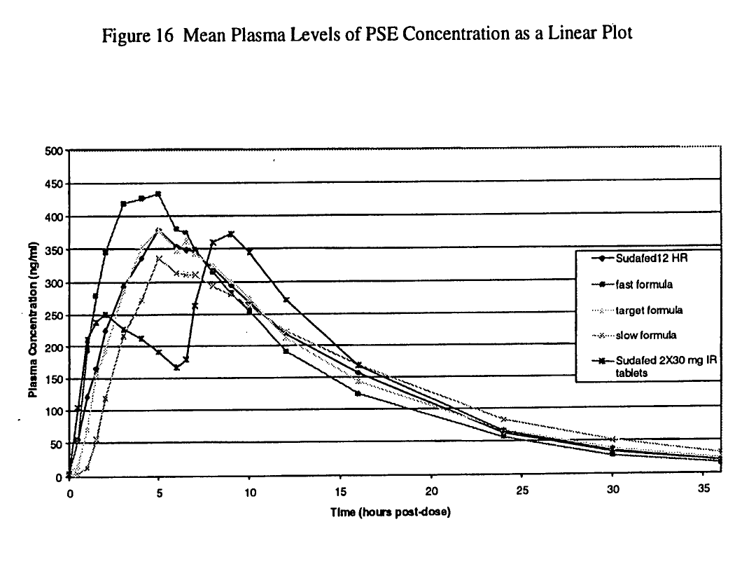

FIG. 16 will demonstrate the in vivo release rates of the fast (6% SR), moderate (10% SR) and slow releasing (14% SR) formulations of the present invention. Additionally this figure will demonstrate the Sudafed 12 Hour caplets and the Sudafed Immediate Release 2×30 mg tablets (60 mg dosed at 6 hours apart).

The linear plot of plasma concentration vs. time shows that of the three PSE 120 mg formulations, the fast releasing (6% SR coat) formulation gave the greatest Cₘₐₓ, followed by the 10% SR coat formulation, followed by the slow 14% SR coat formulation with the lowest Cₘₐₓ. The 10% SR coat formulation profile closely matched that of the Sudafed 12 Hour Caplet.

This figure also demonstrates that each of the three test formulations (fast, target and slow releasing) were bioequivalent to Sudafed 12 Hour Caplets with respect to AUC₍ₒ₋ₜ₎, AUC_{(0-infinity)}, and Cₘₐₓ.

Each of the three SR test formulations (slow, target and fast releasing), were bioequivalent to immediate release Sudafed with respect to AUC₍ₒ₋ₜ₎, AUC_{(0-inf)}, and Cₘₐₓ.

The tₘₐₓ was significantly (p<0.05) shorter for the “fast” formulation compared with Sudafed® 12 Hour Caplets. There was no significant difference between the “slow” and the “moderate” formulations.

The tₘₐₓ for each of the three test formulations was significantly (p<0.0001) delayed compared with the first dose of immediate release Sudafed®.

In FIG. 16, the Comparitor products are Sudafed® 12-Hour 120 mg SR caplet and a Sudafed® immediate release (IR) tablet 2×30 mg. The three SR PSE formula are fast, target and slow as defined herein. The figure provides AUC, Cₘₐₓ and tₘₐₓ parameters:

For the parameter AUC₀₋ₜ (ng.h/ml) the comparison slow PSE formulation vs the SR Comparitor is bioequivalent (CI: 0.91-1.01), with mean of 0.95

For the parameter AUC₀₋ₜ the comparison target PSE formulation vs the SR Comparitor is bioequivalent (CI: 0.92-1.02), with mean of 0.97.

For the parameter AUC₀₋ₜ the comparison fast PSE formulation vs the SR Comparitor is bioequivalent (CI: 0.95-1.05), with mean of 1.0.

For the parameter AUC₀₋ₜ (ng.h/ml) the comparison slow PSE formulation vs the IR Comparitor is bioequivalent (CI: 0.93-1.03), with mean of 0.98.

For the parameter AUC₀₋ₜ the comparison target PSE formulation vs the IR Comparitor is bioequivalent (CI: 0.95-1.05), with mean of 0.99.

For the parameter AUC₀₋ₜ the comparison fast PSE formulation vs the IR Comparitor is bioequivalent (CI: 0.98-1.08), with mean of 1.03.

For the parameter AUC_{0-∞} (ng.h/ml) the comparison slow PSE formulation vs the SR Comparitor is bioequivalent (CI: 0.93-1.03), with mean of 0.98.

For the parameter AUC_{0-∞} the comparison target PSE formulation vs the SR Comparitor is bioequivalent (CI: 0.92-1.03), with mean of 0.98.

For the parameter AUC_{0-∞} the comparison fast PSE formulation vs the SR Comparitor is bioequivalent (CI: 0.95-1.06), with mean of 1.00.

For the parameter AUC_{0-∞} (ng.h/ml) the comparison slow PSE formulation vs the IR Comparitor is bioequivalent (CI: 0.96-1.07), with mean of 1.02.

For the parameter AUC_{0-∞} the comparison target PSE formulation vs the IR Comparitor is bioequivalent (CI: 0.96-1.06), with mean of 1.01.

For the parameter AUC_{0-∞} the comparison fast PSE formulation vs the IR Comparitor is bioequivalent (CI: 0.98-1.08), with mean of 1.03.

For the parameter Cₘₐₓ (ng./ml) the comparison slow PSE formulation vs the SR Comparitor is bioequivalent (CI: 0.843-0.93), with mean of 0.88.

For the parameter Cₘₐₓ the comparison target PSE formulation vs the SR Comparitor is bioequivalent (CI: 0.97-1.08), with mean of 1.02.

For the parameter Cₘₐₓ the comparison fast PSE formulation vs the SR Comparitor is bioequivalent (CI: 1.11-1.23), with mean of 1.17

For the parameter Cₘₐₓ (ng/ml) the comparison slow PSE formulation vs the IR Comparitor is bioequivalent (CI: 0.82-0.91), with mean of 0.87.

For the parameter Cₘₐₓ the comparison target PSE formulation vs the IR Comparitor is bioequivalent (CI: 0.95-1.05), with mean of 1.0.

For the parameter Cₘₐₓ the comparison fast PSE formulation vs the IR Comparitor is bioequivalent (CI: 1.09-1.21), with mean of 1.15.

The establishment of an in vitro-in vivo correlation for PSE was similar to that of CPM and PPA, and in this instance was done by estimating the time course of the fraction of dose absorbed and comparing the transformed data to an in vitro time course of dissolution of the fraction of dose release of the same=formulation. The Wagner-Nelson method, described previously, was used to estimate the fraction of dose absorbed time course in vivo. Averaged subject plasma vs. time data were used for each Clock formulation at each of the nineteen time points and the Wagner-Nelson method was used to estimate fraction of dose absorbed (FA).

PSE exhibits a first order elimination rate constant and along with the blood plasma concentrations and time intervals, were used in the Wagner-Nelson method calculated estimates of the time course of fraction of dose absorbed. Overall subject averages of plasma blood levels vs. time were used to estimate the fraction of dose absorbed when using the Wagner-Nelson calculation.

In order to obtain the best in vitro-in vivo correlation, the PSE 120 mg SR formulations were subjected to different in vitro dissolution conditions. It was found that in-vitro dissolution vs. time profiles of the PSE SR formulations were not significantly affected by the following: changing dissolution media pH, changes in ionic strength, using simulated gastric fluid, changing basket speeds or, by the addition of sodium dodecyl sulfate in the media. The final in vitro condition selected, to achieve the IV/IVC, was 900 ml of 0.1 N HCl as the dissolution media, using USP Apparatus I (baskets) at 100 RPM. These data provided that basis for linear correlation coefficients for each of the three formulations.

Another aspect of the present invention relates to the manufacture of beadlets of Dextromethorphan HBr (DXM) produced by the processes disclosed herein, and to the pellets themselves. The beadlets may be immediate release or sustained release.

As previously noted various admixtures of (DXM) are contemplated with other suitable cough/cold preparations such as the CPM, PPA, and PSE beadlets produced herein (in varying strengths, and amounts).

The solubility of Dextromethorphan HBr in water was found to be 2 g/l (% w/v). The solids concentration of the drug layering solutions in the CPM, PPA, and PSE products herein were found to be about ˜20% w/v. To use a solution for drug layering the Dextromethorphan would require a much more dilute solution that than the other products. The process would take far too long to be commercially feasible. Attempts were made to improve the solubility of the Dextromethorphan HBr by heating the drug layering suspension up to 60° C., adjusting the pH of the solution between 2 and 8 and adding suitable solubilizing agents (Tween 80 and Sodium Lauryl Sulfate). None of these changes to the drug layering preparation improved the solubility of the drug to a degree as to make spraying from a solution feasible.

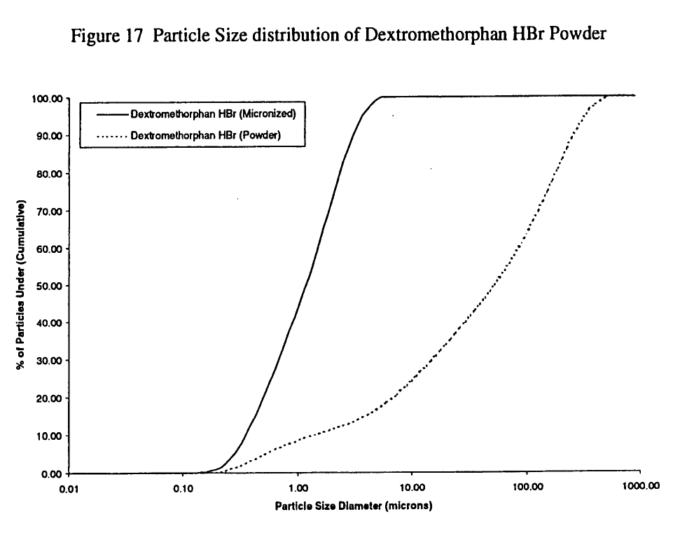

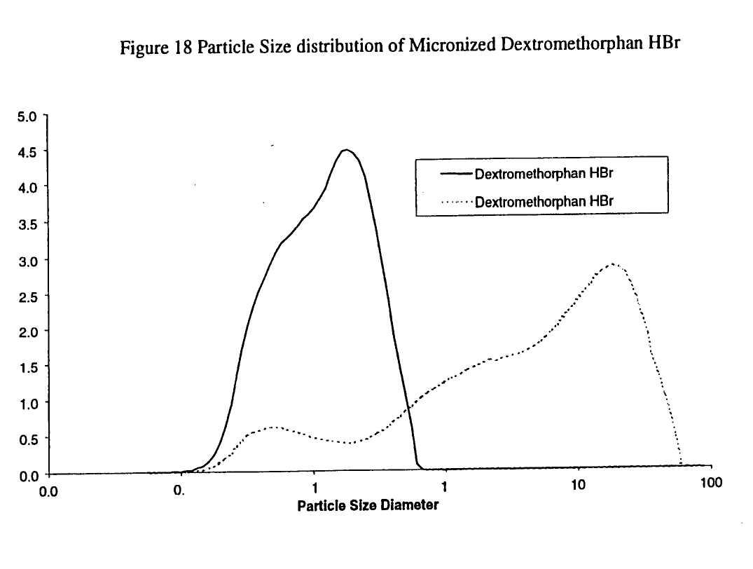

Because of the solubility issue, drug layering proceeded via a suspension coating process. While a number of different process conditions may be utilized a preferred method is to add the drug and suspended it using a lightning type mixer and apply it directly to the sugar spheres. Initially, the Dextromethorphan HBr was added in the form of a powder. The particle size distribution of this powder can be seen in FIG. 17. The process was conducted on the GPCG-1 with a Wurster insert. This process gave poor yields (70% Max). The poor yields were attributed to the relatively large particle size of the drug in comparison to the starting sphere. The drug-layered spheres produced were very rough with large drug particles sticking out from the surface of the pellet. The drug was then micronized using a jet mill to obtain a pulverized drug powder that had a particle size distribution as shown in FIG. 18. FIG. 18 will demonstrate that greater than 90% of the particles are smaller than 5 microns. The micronized drug was then added to the drug suspension and was sprayed with constant mixing. The process was performed on the GPCG 5 with a 9″ Wurster HS insert. This process improved yields into the upper 90%'s. The process with the micronized drug also gave much smoother pellets than the powder process.

Therefore, another aspect of the present invention is the use of micronized DXM for production of a beadlet produced using an aqueous coated processes as claimed herein. Suitable, the DXM particles are less than 50 microns, preferably less than 25, more preferably less than 10, and most preferably less than 5 microns in size. This invention also provides for the micronized DXM particles themselves having a particle size between about 0.1-50 microns.

Process parameters typically are to keep the pellet to suspension particulate to a ratio of 20:1.

30-35 mesh sugar spheres were utilized for the DXM beadlets with a drug load chosen to be ˜50%. However, a drug load could range from 5 to about 90%, preferably 30 to 70, and more preferably 40 to 60%. To adhere the drug to the sugar sphere, hydroxypropylmethyl cellulose (HPMC) was chosen as a suitable binder. A range of drug to binder ratios may be used for this step in the drug layering suspension. They range from 10:0.8, 10:1.0, 10:1.2, 10:1.5, 10:1.8 to 10:2.1. A ratio of 10:1.2 provides for the smoothest, hardest pellets and good yields while minimizing the use of HPMC. All the other ratios with higher levels of HPMC also produce high quality pellets.

For purposes herein (for any active agent), alternative binders may be used, such as but not limited to PVP, HPC, CMC, gum acacia, xanthan, corn syrup, sorbitol, maltitol, or polyvinyl alcohol.

The amount of DXM solids in the drug layering suspension may then be optimized, from about 1 to about 40% being suitable. Preferably, from about 10 to about 30, more preferably about 15 to 25%, with 19% (w/w) as most prefered. Suspensions with greater solids contents (as much as 30%) on became quite viscous and difficult to spray and do not decrease the batch processing time.

Another aspect of the present invention is directed towards a product comprising a sustained release (SR) phase of Dextromethorphan (DXM) beadlets coated with about 0.5 to about 15% (weight gain) of a pseudolatex water swellable polymer dispersion. Preferably, from 3 to 10%, more preferably 4 to 7% and most preferably about 5% weight gain of polymer dispersion.

Another aspect of the present invention is DXM pellets intended for use as an immediate release phase. Dextromethorphan is commonly used in amounts for immediate release of about 5 to 30 mg/dose and in a sustained release about of about 10 to 60 mg/dose. Suitably, the sustained release is over a 12 hour period, although less (8 hours) or more (16 hours) may also be obtained using the process conditions herein. Therefore, another aspect of the present invention is micronized DXM/per dosage form containing from about 5 to 60 mg/dose, be for IR or SR use.

Another aspect of the present invention is the ratio of IR beadlets to SR beadlets of Dextromethorphan. Suitable ratios of IR:SR pellets of DXM are 90:10 to 10:90, 70:30 to 30:70, 60:40 to 40:60, and 50:50 (or 1:1).

Combinations of DXM beadlets with other cough/cold products is also recognized as another aspect of the present invention. Suitable combinations are use with decongestants such as PSE or PPA, and also in combination with an antihistamine such CPM, BPM, or the non-sedating antihistamines. Suitable combinations are PPA (50 to 75 mg)/CPM (4 to 12 mg)/DXM (15 to 30 mg) in dosages of 50 mg/4 mg/15 mg; or a 50 mg/4 mg/30 mg dosage form. For a larger sustained release formulation, a 75 mg/8 mg/60 mg dosage would be suitable. For PSE combinations suitable dosage amounts are PSE (60 to 120 mg)/DXM (15 to 30 mg)/CPM (4 to 12 mg).

It is also contemplated to combine DXM with an NSAID, such as the OTC or Rx available ibuprofen, ketoprofen or naproxen sodium. Also suitable for use are the newer COX-1 or COX-2 active agents, such as Vioxx, or Celebrex. Suitable amounts of ibuprofen are 200 to 1200 mg ibuprofen (12 hour) with 15 to 30 mg DXM.

It is also recognized that various pediatric dosage forms of the above active agents may be formulated herein, such as a 2.5 mg IR DXM with 2.5 mg SR DXM dosage.

The level of a HPMC seal coat for DXM is preferably between 1 and 3%, most preferably about 2%.

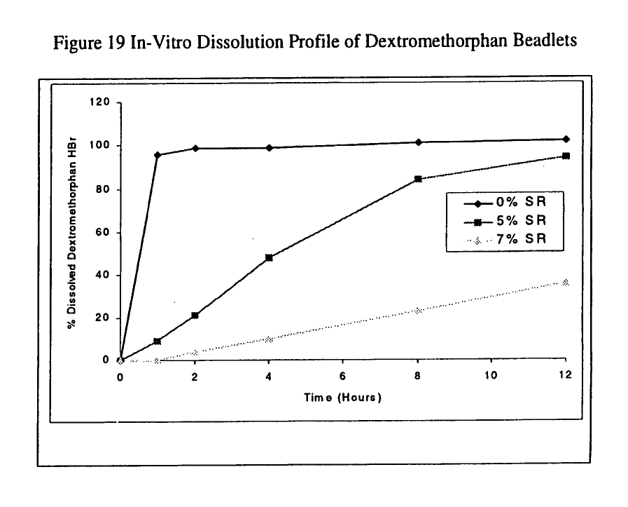

Similar to the other products herein, for the drug layered and sealed pellets of DXM ethylcellulose is the preferred polymer to control the release rate. The target release is bioequivalant to a single active, 15 mg Dextromethorphan HBr immediate release cough syrup dosed at 6 hour intervals. The level of ethylcellulose was evaluated at 0, 5, 7, and 9% and the release profiles were determined in vitro. The three formulations with 0, 5 and 7% SR were chosen as formulations to be dosed in a PK study to obtain blood level data. The in-vitro dissolution profiles can be seen in FIG. 19.

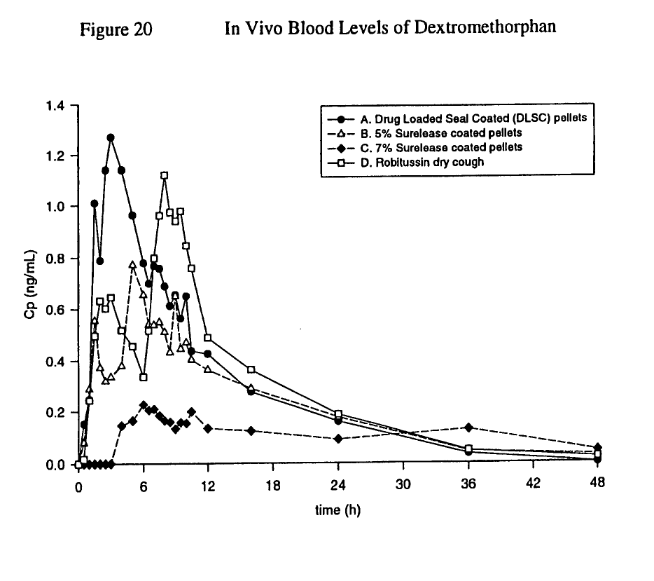

Another aspect of the present invention is the AUC, Cmax, and tₘₐₓ of the IR and 5% and 7% SR pellets of DXM as demonstrated in FIGS. 20 to 22 for dextromethorphan, free dextromethorphan and total dextromethorphan The Comparitor product is the IR liquid, Robitussin Dry Cough Syrup. 10 mg of Robitussin cough syup is dosed at 0 hours and 6 hours, and each 10 ml contains 15 mg dextromethorphan HBr.

The Mean Cₘₐₓ Ratio and 90% Confidence Intervals for the formulations demonstrated in FIG. 20 are shown below in Table 1.

TABLE 1

| Variable | Mean Ratio | Lower 90% CI | Upper 90% CI |
|---|---|---|---|
| IR DXM v | 1.5188 | 0.5941 | 3.8826 |
| Robitussin | | | |
| 5% SR DXM | 0.5449 | 0.2113 | 1.4047 |
| v Robitussin | | | |
| 7% SR DXM | 0.1103 | 0.0429 | 0.2831 |
| v Robitussin | | | |

The Mean AUC₀₋ₜ Ratio and 90% Confidence for the formulations demonstrated in FIG. 20 are shown below in Table 2.

TABLE 2

| Variable | Mean Ratio | Lower 90% CI | Upper 90% CI |
|---|---|---|---|
| IR DXM v | 0.9728 | 0.3717 | 2.5460 |
| Robitussin | | | |
| 5% SR DXM | 0.3826 | 0.1449 | 1.0100 |
| v Robitussin | | | |
| 7% SR DXM | 0.0385 | 0.0146 | 0.1011 |
| v Robitussin | | | |

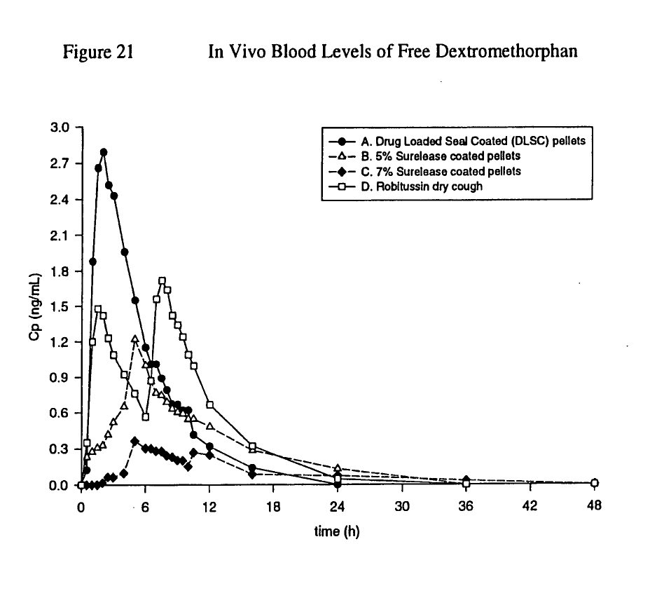

The Mean Cₘₐₓ Ratio and 90% Confidence Intervals for the formulations demonstrated in FIG. 21 for Free Dextromethorphan are shown below in Table 3.

TABLE 3

| Variable | Mean Ratio | Lower 90% CI | Upper 90% CI |
|---|---|---|---|
| IR DXM v | 1.5689 | 1.2399 | 1.9851 |
| Robitussin | | | |
| 5% SR DXM | 0.6711 | 0.5292 | 0.8509 |
| v Robitussin | | | |
| 7% SR DXM | 0.2470 | 0.1940 | 0.3145 |
| v Robitussin | | | |

The Mean AUC₀₋ₜ Ratio and 90% Confidence Intervals for the formulations demonstrated in FIG. 21 for Free Dextromethorphan are shown below in Table 4.

TABLE 4

| Variable | Mean Ratio | Lower 90% CI | Upper 90% CI |
|---|---|---|---|
| IR DXM v | 1.0266 | 0.7199 | 1.4640 |
| Robitussin | | | |
| 5% SR DXM | 0.6891 | 0.4817 | 0.9859 |
| v Robitussin | | | |
| 7% SR DXM | 0.1570 | 0.1090 | 0.2260 |
| v Robitussin | | | |

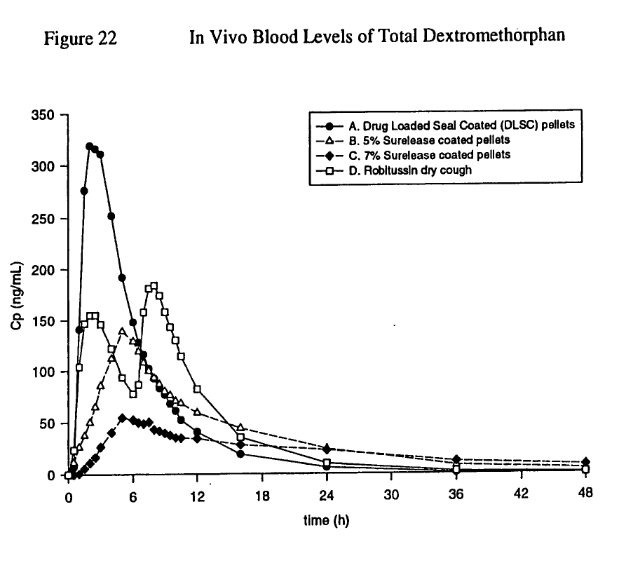

The Mean Cₘₐₓ Ratio and 90% Confidence Intervals for the formulations demonstrated in FIG. 22 for total dextomethorphan are shown below in Table 5.

TABLE 5

| Variable | Mean Ratio | Lower 90% CI | Upper 90% CI |
|---|---|---|---|
| IR DXM v | 1.7549 | 1.4842 | 2.0760 |
| Robitussin | | | |
| 5% SR DXM | 0.7484 | 0.6320 | 0.8862 |
| v Robitussin | | | |
| 7% SR DXM | 0.2840 | 0.2400 | 0.3361 |
| v Robitussin | | | |

The Mean AUC₀₋ₜ Ratio and 90% Confidence Intervals for the formulations demonstrated in FIG. 22 for total dextomethorphan are shown below in Table 6.

TABLE 6

| Variable | Mean Ratio | Lower 90% CI | Upper 90% CI |
|---|---|---|---|
| IR DXM v | 1.0572 | 0.9371 | 1.1926 |
| Robitussin | | | |
| 5% SR DXM | 0.8942 | 0.7918 | 1.0099 |
| v Robitussin | | | |
| 7% SR DXM | 0.5260 | 0.4660 | 0.5937 |
| v Robitussin | | | |

The Median Tₘₐₓ Difference and 95% Confidence Intervals for the formulations demonstrated in FIG. 22 for total dextomethorphan are shown below in Table 7.

TABLE 7

| | | Lower | Upper | |
|---|---|---|---|---|
| Variable | Median | 95% CI | 95% CI | p value |
| IR DXM v | 5.500 | 3.983 | 5.999 | 0.0018 |
| Robitussin | | | | |
| 5% SR DXM | 2.517 | 0.984 | 3.000 | 0.0172 |
| v Robitussin | | | | |
| 7% SR DXM | 1.500 | −0.016 | 2.501 | 0.0599 |
| v Robitussin | | | | |

A positive value for median difference indicates that the test product has a faster Tₘₐₓ than the reference product.

Similar to the other products herein, a top coat is applied to the beadlets containing a coating of Surelease® in the final product. The ethylcellulose coating needs to be “cured” after it is applied to the pellets. This curing ensures that the coat has polymerized completely and is uniform. In order to cure the product the temperature must be raised to about 60° C. and held there for about 1 hour. At this temperature, the ethylcellulose (in Surelease®) is above its' glass transition temperature (t_{g}) and is in a rubbery state and rather “sticky”. The product cannot be cured with Surelease on the outside of the pellet because all the pellets would stick together while in this rubber state. A top coat provides an outer layer that allows the product to be cured while maintaining discrete, fluidized particles in the coating unit.

Therefore, another aspect of this invention is the need, and length of time for curing of final products having the desired characteristics. The dissolution profile is stablized and provides for a uniform membrane or completely coalesed film surrounding the beadlet/pellet. It has now been found that for ethylcellulose that a suitable time period is about 60 minutes, at a temperature about or above 60° C.

While a number of top coats well known in the art can be used, a suitable top coat for the DXM beadlets was considered to be a hydroxypropylmethylcellulose based product, such as Colorcon Opadry® Pink.

Levels for Top Coat and Drug Weight were determined and the top coat level is taken from the other pellets (CPM, PPA, PSE). The Drug Weight represents a 1:1 ratio of drug to sugar sphere. The remaining % is the binder and seal coat.

Ethylcellulose is the functional barrier coat (sustained release coat) common to all the formulations herein More specifically, an aqueous pseudo-latex of ethylcellulose is preferred (trade name Surelease E-7-19010), as this product provided the best ease of processing and drug release reproducibility. In such a system, changing the level of ethylcellulose coat will most easily modulate drug release. Changing the pellet drug load, to a lesser extent, can also modulate the rate of drug release from such coated pellet drug delivery systems. The levels of water-soluble coats, such as hydroxypropylmethylcellulose (HPMC) seal coat and the Opadry® color topcoat do not substantially affect the rate of drug release and cannot be used as a drug release rate modulator in this system.

One aspect of the present invention is the optimization of parameters involved in aqueous coating techniques for use on water soluble active agents. It is an unexpected finding that this technology can be applied to active agents which would readily dissolve during the manufacturing process. It has now been found that by use of tightly held procedures as described herein that products, containing a water soluble dispersion for either sustained release or immediate release may be produced.

Development of a manufacturing procedure to produce IR and SR beads or pellets that are physically, chemically and therapeutically equivalent to currently marketed pharmaceutical standards is one use of the process described herein. The ability to develop a new manufacturing procedure for production of IR and SR beads of new marketed products is another aspect of this invention.

One drawback to global manufacturing procedures has been the use of ingredients, and processing conditions used to manufacture the pellets or beads which do not meet each countries standards and local market regulations. It appears that use of the preferred polymer coating herein, ethylcellulose, and the process conditions described are suitable for world wide manufacturing.

The process herein uses fluid bed coating equipment based upon the Wurster design, also called a Wurster column. This process is considered the best suited to coating of beads or beadlets. It is the industry standard and widely accepted in many companies around the globe. Wurster columns provide more efficient drying that other coating units which results in higher spray rates, faster processing (shorter processing time) and high quality finished products.

It consists of two columns, one inside the other. The air-flow pattern is such that most of the air flows through the inside column. This facilitates the beadlets movement in an upward direction inside the smaller column and in a downward direction in the space between the columns. The spraying nozzle is located at the bottom in the middle of the smaller column so that the beadlets are coated when they travel up and dry outside the smaller column in the space between the two columns. The geometric proportions of the inner and outer columns are such that a continuously moving column of beads or tablets passes through the spray path with every tablet/bead capturing some of the coating, and at the same time, ensuring that little or no solution reaches the wall of the inner column.

Smaller model are usually equipped with either 6- or 12-inch diameter outer chambers (having capacities in the range of 1-2 kg and 10-15 kg, respectively), production models are usually based on an 18-inch chamber diameter. Any attempt to increase the diameter while retaining only a single spraygun usually results in some beads passing through the spray gun without receiving any coating. Consequently, larger models use multiples of the 18-inch concept; for example, the 32-inch model has 3 inner coating partitions and sprayguns, while the 46 inch model has seven, all based on the 18-inch geometry, allowing for capacities up to 400 kg.

The actives and excipients can be delivered to the spraying zone as solutions, suspensions, emulsions or melts. The most acceptable processing solvent for processes and coatings is water. The cost is minimal, and there are no environmental considerations.

It is important to have the liquid in atomized state prior to contact between the liquid particle and the beadlets (substrate). Large droplet size can cause agglomeration and reduce the yield.

The liquid delivery system consists of pumps and spraying nozzles. The liquids are atomized when they leave the nozzles by compressed air running into the nozzle simultaneously with the liquid. The higher the atomization air pressure the smaller is the atomized droplet. Peristaltic pumps are the most widely accepted for liquid delivery systems because of their accuracy and conformity to GMP requirements.

The major principal of the technology is uniform liquid delivery and controlled evacuation of the moisture from the product/beadlet during processing. An equilibrium must be maintained through the entire coating process to prevent agglomeration (when evaporation is inadequate) or spray drying (when evaporation is too fast).

The following are “industry accepted” critical process parameters for Fluid Bed Technology and Wurster coating processes.
Product Temperature
Spray Rate
Inlet Air Flow (Velocity)
Inlet Air Temperature
Atomization Air Pressure
Atomization Air Flow
Inlet Air Humidity; and
Bead Surface

The most important processing parameters are Product Temperature and Spray Rate. All other parameters can be derived from these parameters. Product Temperature is important for maintaining a drying environment, surface characteristics (the surface porosity and uniformity/sphericity depends on the rate of crystallization and film forming properties and maintaining the product above/below the glass transition point of the polymers used in the coating process.

Spray Rate is an important factor for achieving the correct processing environment. i.e. rate of evaporation. This parameter can also influence surface characteristics (the surface porosity and uniformity/spherosity depends on the rate of crystallization and film forming properties of the material being applied). When Spray Rate is balanced with the desired product temperature, the product can be maintained at the selected “Steady-State” conditions.

Inlet Airflow and Inlet Air Temperature are also important factors, however they are derivatives of Product Temperature and Spray Rate. The Inlet Airflow and Inlet air temperature provide energy to the coating process by means of “conditioned” fluidization air. This conditioned air provides the correct thermodynamic equilibrium so that the product is not under/over-wet during processing.

The present invention has determined optimal ranges of product temperature, spray rates for different liquids, air flows and inlet air temperatures to provide necessary product temperature ranges at different spray rates, atomization air pressures to accommodate different spray rates of liquids used for coatings, and the significance of inlet and exhaust air humidity for coating processing for a select group of actives and coating polymers. Use of this information will enable the skilled artisan to readily apply this data to other water soluble actives with ease and to work with other water soluble polymers for coating said active ingredients.

It has been determined that optimal sustained release coating agents should not require the addition of significant amounts of suspending agents, such as talc which are dispersed in the coating liquids. For instance, Eudragit™ coatings require talc to reduce tackiness of the polymer during the coating process. Rigorous mixing with a homogenizer during the liquid preparation is necessary. The presence of talc presents the potential for nozzle clogging. Talc also creates more dust in the fluid bed during the coating process that affects the appearance of the bead surface.

Suitably, the sustained release coating agents is one which is useful world wide, is compatible with water as a solvent, is environmentally friendly, easy to use, and provides a stable product. Preferably, the coating is ethyl cellulose. The preferred delivery of ethyl cellulose is a pseudolatex ethyl cellulose dispersion, as ethyl cellulose is insoluble in water. Such dispersions are produced by multiple manufactures, FMC and Colorcon. The FMC product uses dibutyl sebecate as a plasticizer where as the Colorcon product uses a medium chain triglyceride, coconut oil. While the Colorcon product is preferred, it is a suitable alternative to utilize a different manufacturer's ethylcellulose dispersion with modifications, such as addition of vegetable oils, lecthicins, or citrate salts.

During evaluation of the coating process, it was determined that a seal or barrier coat between the drug layer and the sustained release coating is essential to provide a consistent release profile from the beads. Preferably the seal coat is from about 1 to 12% weight gain, depending upon the ability of the drug to migrate. In the case of CPM and PPA herein a 1 to 7%, more preferably about a 2-5% weight gain is utilized, respectively. Weight gain as used herein means the amount of solids added to a pellet at that particular coating stage.

The amount of sustained release coating and color coating applied to the beadlets can be problematic. Higher concentrations of the latex dispersion requires careful monitoring and control of product temperature and solution spray rate during transition from sustained releaser to the color coating to ensure that the beadlets do not agglomerate and stall in the fluidized bed.

A minimum of 3.7 bar steam is required to maintain adequate in-let air temperature in a Glatt fluidized bed processing machine. Control of in-let air temperature and bed temperature is critical during application of the ethylcellulose coating. A high in-let air temperature (>70° C.) will cause the bottom plate of the Glatt to over heat. The sustained release coated beadlets will stick and agglomerate on the hot bottom plate and stall the bed.

The spray rate for the sustained release should also be conservative, starting with 450 gms/min and increasing to about 850 gms/min. The relatively slow spray rate is to allow for a lower in-let air temperature and formation of fine droplets to produce a smooth film on the beads.

The transition temperature from SR coating to color coating of the in-let air must be brought up slowly to achieve drying of the color solution but not too hot as to cause agglomeration of the SR coated beads. The initial stages of color coating is especially sensitive. The in-let air temperature should not exceed about 65° C. and application of the color coating should begin as soon as the bed temperature reaches about 45° C. After 30 minutes into the color coating, an increase in the spray rate to 750 gm/min may be made with adjustment of the in-let air temperature such that the bed temperature is maintained at about 40 to about 50° C.

An important feature of this process is the use of the glass transition temperature of the water swellable polymer. Above this high temperature, the film will become tacky, and beadlet agglomerates will form. This will result in loss of beadlets due to screening/sizing and require a higher amount of polymer to be applied. The product appearance and drug content will vary, and produce unsatisfactory product.

It has now been found that the glass transition temperature of Surelease® is about 38° C. to about 41° C., (closer to 39-41° C.) dependent somewhat upon the amount of dilution with water. Therefore, it is relatively safe to conduct the process at 43±4° C. at the beginning and then reduce the Product temperature to a steady state target of about 37 (±4)° C. Depending upon suitable changes in the equipment, scale of commercial reaction process and amount of functional coating agent, the skilled artisan may find that the temperature range can be expanded to 38° C. to about 41°+/−2° C. Product temperature is the actual temperature of the beadlet in the Wurster column. The relatively higher initial Product temperature is to prevent water soluble drug penetration into the SR coating layer. If the coating process has a reduced spray rate the product temperature may increase above 45° C. This is above the glass transition temperature of Surelease®.

The process of the present invention requires coating the drug loaded, and sealed pellet with the aqueous water swellable polymeric dispersion. Initially the coating of the polymeric dispersion is at a product temperature above the glass transition point of the polymeric dispersion. The temperature of the product is then lowered to below the glass transition point of the polymeric dispersion and maintained at a steady state temperature after a sufficient amount of the water swellable polymeric dispersion has been applied. Although the skilled artisan would readily recognize when a sufficient amount of polymer has been applied, about a 30 minute spraying time, after about a ½% of weight gain of polymer has been applied is deemed a “sufficient amount”. The purpose of a “sufficient amount” is to create a protective layer of functional coat to prevent water getting into the seal coat.

It is recognized that in-let air temperature and in-let air flow are determined from the two parameters, spray rate and product temperature. They are also determined by the size of the equipment and the type of means by which the air is heated. For instance, in a small Wurster unit (15/12″) the in let air temperature may range form about 64 to about 109° C. For a 60/18″ from about 55 to about 89; and in a 120/32″ from about 70 to about 100° C. Similarly in-let air flow may vary from 600, to 1150-3000 and from 2400-3300 respectively. The calculation and control of these parameters is well within the skilled artisan's means knowing the spray rate and product temperature. Air flow is important to maintain for two reasons: it provides sufficient fluidization and enough energy to evacuate the moisture without resorting to high Inlet Air Temperature in the chamber.

The product temperature is controlled by the inlet air flow and temperature, it is preferably maintained within +/−4° C. of the target temperature, more preferably +/−1.5° C. of the target temperature during the steady state process.

The product temperature during the initial application stage of the water swellable polymer application, (about 30 minutes) should be maintained above the glass transition point temperature. During the rest of the application, the temperature may be maintained at below the glass transition point.

The product temperature during the initial stage of top coating is preferably maintained below the glass transition point. The temperature may be increased above the glass transition point about 30 minutes into the top coat application.

Another important parameter of this process is dew point. A lower dew point will produce drier air. This will reduce drug mobility through the functional coat, such as the ethylcellulose layer. It will also reduce the tackiness, which develops in the ethyl cellulose dispersion application. If the dew point is to high moisture will be entrapped in the pellet as layers of the SR coating are applied. During the curing process molecules of water, i.e. moisture, will remain entrapped in the beadlet and produce a product which will not meet specifications. For purposes herein, dew point is preferably maintained at about 8+/−3° C. to about 11+/−3° C. (or 9+/−5° C.) with 9+/−3° C. preferred for conditioned/filtered/chilled/air. More preferably, the dew point is sharply maintained with a small variation of about +/−1°, if possible.

The dew point may be varied depending upon the stage in the manufacturing process, for instance Drug layering as opposed to functional coating. It may also very within the broader parameters by the active agent. Drug layering (for all actives) requires tighter controls on dew point, hence the 9+/−5° C. noted above. However, for functional coating the dew point may be broaded to 5° C. to 20° C. In particular for CPM, 5° C. to 20° C. is suitable, with 14+/−6 better, and 15+/−3° C. more preferable. PSE produces a better product at the 9+/−6, with 9+/−3 being preferable. As long as the thermodynamic equilibrium is present, and the drying is sufficient, there is no large effect of the DEW point variable.

The invention will now be described by reference to the following examples which are merely illustrative and are not to be construed as a limitation of the scope of the present invention.

The following examples will demonstrate the production of drug loaded pellets with two different active ingredients, chlorpheramine maleate and phenylpropanolamine. The drug load pellets are then utilized to make either immediate release beads (IR) or sustained released beads (SR). The 4 populations of beads are then filled into capsules at a predetermined ratio.

### EXAMPLE 1

### General Process—PPA Drug Load Pellets

In this example phenylpropanolamine HCl, as the active ingredient, is loaded onto 30-35 mesh Sugar spheres, layered in GPCG fluidized bed unit, seal coated and screened through #20 and #30 mesh screens.

RAW MATERIAL DATA

| | | Quantity per |
|---|---|---|
| Ingredient | %, w/w | Batch (Kg) |
| Purified Water 1 | — | 1.32 |
| HPMC E5 USP/EP | 1.35 | 0.014 |
| Phenylpropanolamine | 47.62 | 0.48 |
| Hydrochloride | | |
| Non Pareil Seeds 30-35# USP/NF | 46.27 | 0.46 |
| Opadry White | 4.76 | 0.048 |
| TOTAL | 100.00 | 1.00 |

This example will produce 50 mg of Phenylpropanolamine Hydrochloride per capsule.

MANUFACTURING FORMULAS
HPMC, 10% Solution

| | | Quantity per |
|---|---|---|
| Ingredient | %, w/w | Batch (Kg) |
| Purified Water | 90.00 | 0.12 |
| Methocel, NF | 10.00 | 0.01 |
| TOTAL | 100.00 | 0.13 |

Phenylpropanolamine HCL Solution

| | | Quantity per |
|---|---|---|
| Ingredient | %, w/w | Batch (Kg) |
| Purified Water | 55.06 | 0.76 |
| Phenylpropanolamine HCL, USP | 35.00 | 0.49 |
| Powder | | |
| Methocel, NF 10% Solution | 9.94 | 0.14 |
| TOTAL | 100.00 | 1.39 |

Opadry Dispersion

| | | Quantity per |
|---|---|---|
| Ingredient | %, w/w | Batch (Kg) |
| Purified Water | 90.00 | 0.43 |
| Opadry White | 10.00 | 0.05 |
| TOTAL | 100.00 | 0.48 |

Phenylpropanolamine HCl and Opadry,
10% Dispersion (Seal Coat) Layering

| | | Quantity per |
|---|---|---|
| Ingredient | %, w/w | Batch (kg) |
| Non Pareil Seeds 30-35# USP/NF | 19.89 | 0.46 |
| Phenylpropanolamine Hydrochloride, | 59.65 | 1.39 |
| 35% Solution* | | |
| Opadry White. 10% Dispersion | 20.47 | 0.48 |
| TOTAL** | 100.00 | 2.33 |

*Water evaporates during the processing.
Total solids content is about 36%. (Solids total consists of Phenylpropanolamine HCl and HPMC E5 USP/EP)

Processing
HPMC E5, 10% Solution

Pursuant to directions of the manufacture of the particular brand of HPMC, heat 0.062 kg of Purified Water to 70±10° C. Add HPMC E5 USP/EP, and mix for 15 minutes (until visually dissolved). Add 0.062074 kg of cold Purified Water, and mix for 15 minutes (until visually dissolved). Allow the solution to cool and de-aerate if desired. The HPMC E5 USP/EP 10% Solution will be used for the manufacturing of the Phenylpropanolamine HCl (PPA).

Phenylpropanolamine HCl 35% Solution

1. Weigh out Purified Water into stainless steel container with a Lightnin' Mixer.
2. Heat the water to 65±5° C.
3. Turn on the mixer. Adjust the air pressure to form a vortex.
4. Charge the Phenylpropanolamine HCl (PPA) into the vortex.
5. When all the Phenylpropanolamine HCl (PPA) is charged allow the solution to mix for 15 (±5) minutes.
6. Add HPMC E5 USP/EP 10% solution to the Phenylpropanolamine HCl (PPA) solution.
7. Continue mixing.
   Opradry White Dispersion
Prepare according to manufacturer's directions.
Weigh out Purified Water into stainless steel container with a Lightnin' mixer. Add Opadry White. Mix for 65 (±5) minutes. Continue mixing until ready to use and through the spraying.

### Fluidized Bed Processing

Phenylpropanolamine HCl and Seal Coat Layering

1. Adjust the inlet air temperature as necessary to control the product temperature.
2. Machine Configuration: GPCG
3. Weigh Non Pareil Seeds #25-30 mesh into a suitable stainless steel container and charge into fluidized bed processor.

Set up the process parameters and conduct the processing as per following recipe:

| | Process Parameters: | | | |
|---|---|---|---|---|
| | Min. | Setup | Max | Unit |
| Temp. Inlet Air | 55.0 | | 75.0 | ° C. |
| Temp. Product | 35.0 | | 55.0 | ° C. |
| Spray Rate | 1.0 | | 10.0 | g/min |
| Prod. Filter Shaking Active | | Off | | s |
| Prod. Filter Shaking Pause | | Off | | s |
| Prod. Filter Shaking Mode | | GPCG | | |
| Temp. Exhaust Air | 30.0 | | 55.0 | ° C. |
| Time | | | | min |

Begin fluidization process. Begin spraying the drug solution. PPA 35% solution until complete using the above noted process parameters.

Connect liquid pumping system to the seal coat solution (Opradry White dispersion solution) and continue spraying until complete.

Screen the batch through the sifter fitted with 20 and 30 mesh screens.

### EXAMPLE 2

### General Process (PPA SR Beads)

This example is directed a process for making phenylpropanolamine HCl sustained release beads (PPA SR) having a 12% coating of Surelease.

The Phenylpropanolamine HCL is layered on to the sugar spheres, seal coated, and sifted as described above in Example 1. The Drug Loaded pellets are charged into a GPCG fluidized bed unit. Sustained Release coat and Top coat as described herein are put on the pellets. The product is cured, and the finished product is screened through #20 and #30 mesh screens.

RAW MATERIAL DATA

| | | Quantity per |
|---|---|---|
| Ingredient | %, w/w | Batch (Kg) |
| PPA Drug layered Pellets of | 86.24 | 0.86 |
| Example 1 | | |
| Surelease ® | 11.76 | 0.47 |
| Opadry AMB | 2.00 | 0.02 |
| TOTAL | 100.0000 | 1.35 |

This will produce 50 mg of Phenylpropanolamine Hydrochloride per capsule

MANUFACTURING FORMULAS
Surelease, 15% Dispersion

| | | Quantity per |
|---|---|---|
| Ingredient | %, w/w | Batch (Kg) |
| Surelease (Colorcon) | 60.00 | 0.47 |
| Purified Water | 40.00 | 0.31 |
| TOTAL | 100.00 | 0.78 |

OPADRY AMB, 10% DISPERSION

| | | Quantity per |
|---|---|---|
| Ingredient | %, w/w | Batch (Kg) |
| Opadry AMB | 10.000 | 0.001 |
| Purified Water | 90.00 | 0.179 |
| TOTAL | 100.00 | 0.199 |

Processing
Surelease, 15% Solids Dispersion
1. Charge Surelease in container equipped with Lightnin' Mixer.
2. Turn on the mixer in the Surelease container. Adjust the compressed air for adequate mixing.
3. Charge the Purified Water into the Surelease container.
4. Mix for 20±5 minutes.
   Opadry 10% Dispersion
Mix in accordance with the Manufactures directions.
Weigh out Purified Water into stainless steel container equipped with a Lightnin' Mixer. Add Opadry Pink. Mix for 65 (±5) minutes. Reduce the air pressure to the dispersion to de-aerate. Continue mixing until ready to use and through the spraying.

### Fluidized Bed Processing

Surelease, 15% Solids (Sustained Release Dispersion) and Opadry, 10% Dispersion Layering

1. Machine Configuration: GPCG
2. Weigh PPA Seal coated Pellets of Example 1 and charge into the fluidized bed.

3. Set up the process parameters and conduct the manufacturing as per following recipe:

| | Process Parameters: | | | |
|---|---|---|---|---|
| | Min. | Setup | Max | Unit |
| Temp. Inlet Air | 55.0 | | 75.0 | ° C. |
| Temp. Product | 35.0 | | 55.0 | ° C. |
| Spray Rate | 1.0 | | 10.0 | g/min |
| Prod. Filter Shaking Active | | Off | | s |
| Prod. Filter Shaking Pause | | Off | | s |
| Prod. Filter Shaking Mode | | GPCG | | |
| Temp. Exhaust Air | 30.0 | | 55.0 | ° C. |
| Time | | | | min |

Begin fluidization process. Begin spraying the Surelease 15% solids dispersion until complete using the above noted process parameters. During the initial spraying of the SR coating, the initial product temperature is higher than the glass transition point to get rapid drying. Once sufficient coating of the polymer is applied, the product temperature is decreased to below the glass transition point to avoid agglomeration. The Inlet Air flow is adjusted as necessary.

Connect liquid pumping system to the top coat solution (Opradry Pink dispersion) and continue spraying until complete. The product temperature is maintained below the glass transition point at steady state, adjusting the inlet air flow rate as necessary.

The pellets are cured, and the batch is screened through # 20 and # 30 mesh screens.

### EXAMPLE 3

### General Process—PPA IR Beads

The following example utilizes the drug layered PPA beads of Example 1.

The process for manufacture of the IR pellets is identical to that of the SR pellets as exemplified in Example 2 above, except for the quantity of Surelease applied. In this instance the IR beadlets have a 4% coating of Surelease applied.

The amounts of Surelease applied is:

RAW MATERIAL DATA

| | | Quantity per |
|---|---|---|
| Ingredient | %, w/w | Batch (Kg) |
| PPA Drug layered Pellets | 94.00 | 0.94 |
| Surelease ® | 4.00 | 0.16 |
| Opadry AMB | 2.00 | 0.02 |
| TOTAL | 100.0000 | 1.12 |

### EXAMPLE 4

### General Process (CPM Drug Loaded and Seal Coated Beads)

Similar to that for PPA described above in Example 1, CPM is loaded onto sugar spheres and used in the IR and SR preparation of Beadlets described in the Examples below.

30-35 mesh Sugar spheres are layered with Chlorpheniramine Maleate in a GPCG fluidized bed unit, seal coated and screened through #20 and #30 mesh screens.

RAW MATERIAL DATA

| | | Quantity per |
|---|---|---|
| Ingredient | %, w/w | Batch (Kg) |
| Purified Water | | 0.68 |
| Non Pareil Seeds #25-30 mesh | 87.92 | 0.88 |
| HPMC E-5 Premium, NF* | 2.28 | 0.02 |
| Chlorpheniramine Maleate* | 9.80 | 0.099 |
| TOTAL | 100.00 | 1.00 |

This process produces 4 mg of Chlorpheniramine Maleate per capsule.

MANUFACTURING FORMULAS
HPMC, 10% Solution

| | | Theoretical |
|---|---|---|
| Ingredient | %, w/w | Batch (Kg) |
| Purified Water | 90.00 | 0.21 |
| HPMC E-5 Premium. NF | 10.00 | 0.023 |
| TOTAL | 100.00 | 0.23 |

CHLORPHENIRAMINE MALEATE SOLUTION

| | | Quantity per |
|---|---|---|
| Ingredient | %, w/w | Batch (Kg) |
| Purified Water | 78.70 | 0.47 |
| Chlorpheniramine Maleate | 16.59 | 0.10 |
| HPMC E-5 Premium. 10% Solution | 4.71 | 0.03 |
| | 100.00 | 0.60 |

### Processing

HPMC E5, 10% Solution

The solution is produced in accordance with manufacturer directions and similar to that in the above examples.
1. harge 0.58625 kg of Purified Water into a stainless steel container equipped with a Lightnin' Mixer. Turn the mixer on. Adjust the air pressure to 3.5-4.5 bars (50-65 lb_{f}/in²) to form a vortex. Heat up the Purified Water to 65±5° C.
2. Weigh out 0.111875 kg of Purified Water from step 1 into stainless steel container equipped with a Lightnin' Mixer.
3. Cool the rest of the hot water in the stainless steel container to 40±5° C. Continue mixing. This portion of the Purified Water will be used for the manufacturing of the Chlorpheniramine Maleate Solution.
4. Turn the mixer in the stainless steel container on. Adjust the compressed air pressure to 60-75 lb_{f}/in² (4-5 bars) to create vortex.
5. Add HPMC E5 into the vortex in the stainless steel container.
6. Mix for 15 minutes (until visually dissolved).
7. Add 0.09375 kg of Purified Water.
8. Mix for 15 minutes (until visually dissolved).
9. Turn OFF the mixer and allow the solution to cool and de-aerate. The HPMC E5 USP/EP 10% Solution will be used for the manufacturing of the Chlorpheniramine Maleate solution and for Seal Coat Solution. The solution can be used for the manufacturing of the Chlorpheniramine Maleate solution prior to completion of de-aeration and cooling.
   Chlorpheniramine Maleate Solution
1. Charge the Chlorpheniramine Maleate into stainless steel container from step 3, HPMC E5, 10% Solution.
2. When all the Chlorpheniramine Maleate is charged allow the solution to mix for 15 (±5) minutes.
3. Add 0.0284375 kg of HPMC E5 10% solution to the Chlorpheniramine Maleate solution.
4. Mix for 5-10 minutes.

### Fluidized Bed Processing

Chlorpheniramine Maleate and Seal Coat Layering

1. Machine Configuration: GPCG
2. Weigh Non Pareil Seeds #25-30 mesh into a suitable stainless steel container.

3. Set up the process parameters and conduct the processing as per following recipe:

| | Process Parameters: | | | |
|---|---|---|---|---|
| | Min. | Setup | Max | Unit |
| Temp. Inlet Air | 55.0 | | 75.0 | ° C. |
| Temp. Product | 35.0 | | 55.0 | ° C. |
| Spray Rate | 1.0 | | 10.0 | g/min |
| Prod. Filter Shaking Active | | Off | | s |
| Prod. Filter Shaking Pause | | Off | | s |
| Prod. Filter Shaking Mode | | GPCG | | |
| Temp. Exhaust Air | 30.0 | | 55.0 | ° C. |
| Time | | | | min |

Begin fluidization process. Begin spraying the drug solution, CPM SOLUTION until complete using the above noted process parameters.

Connect liquid pumping system to the seal coat solution (HPMC 10% Solution) and continue spraying until complete.

Screen the batch through the sifter fitted with 20 and 30 mesh screens.

### EXAMPLE 5

### General Process (CPM SR Beads)

Similar to the IR process below, the Chlorpheniramine Maleate layered, seal coated and sifted Pellets of Example 4 are charged in GPCG fluidized bed unit. Sustained Release coat and Top coat are put on the Pellets. The amount of sustained release coating is 8% Surelease. The product is cured, and the finished product is screened through #20 and #30 mesh.

RAW MATERIAL DATA

| | | Quantity per |
|---|---|---|
| Ingredient | %, w/w | Batch (Kg) |
| Purified Water | | 0.39 |
| CPM drug loaded, seal coated | 90.16 | 0.90 |
| Pellets of Example 6 | | |
| Surelease | 7.84 | 0.31 |
| Opadry Yellow | 1.99 | 0.02 |
| | 100.00 | 1.23 |

Process produces 4 mg of Chlorpheniramine Maleate per capsule.

Surelease, 15% Dispersion

| | | Theoretical |
|---|---|---|
| | | Quantity per |
| Ingredient | %, w/w | Batch (Kg) |
| Surelease | 60.00 | 0.31 |
| Purified Water | 40.00 | 0.21 |
| TOTAL | 100.00 | 0.52 |

Opadry Yellow, 10% Dispersion

| | | Quantity per |
|---|---|---|
| Ingredient | %, w/w | Batch (kg) |
| Opadry Yellow | 10.00 | 0.02 |
| Purified Water | 90.00 | 0.18 |
| TOTAL | 100.00 | 0.20 |

### Processing

Surelease, 15% Solids Dispersion

1. Charge Surelease in container equipped with Lightnin' Mixer.
2. Turn on the mixer in the Surelease container. Adjust the compressed air pressure to 30-45 lb_{f}/in² (2-3 bars) for adequate mixing.
3. Charge the Purified Water into the Surelease container.
4. Mix for 20±5 minutes.
   Opadry Yellow, 10% Dispersion
1. Weigh out Purified Water into stainless steel container equipped with a Lightnin' Mixer,
2. Turn on the mixer, adjust the air pressure to 3.5-4.5 bars to form a vortex.
3. Add Opadry Yellow into the vortex. Avoid splashing and excess foaming. Mix for 35 (±5) minutes.
4. Reduce the mixer air pressure to 2.5-3.5 bars and allow the dispersion to de-aerate.
5. Continue mixing until ready to use and through the spraying.

### Fluidized Bed Processing

Surelease 15% Solids Dispersion (Sustained Release Dispersion) and Opadry Yellow 10% Dispersion (Top Coat) Layering

1. Machine Configuration: GPCG
2. Weigh CPM Seal coated Pellets into a suitable stainless steel container.

3. Set up the process parameters and conduct the manufacturing as per following recipe:

| | Process Parameters: | | | |
|---|---|---|---|---|
| | Min. | Setup | Max | Unit |
| Temp. Inlet Air | 55.0 | | 75.0 | ° C. |
| Temp. Product | 35.0 | | 55.0 | ° C. |
| Spray Rate | 1.0 | | 10.0 | g/min |
| Prod. Filter Shaking Active | | Off | | s |
| Prod. Filter Shaking Pause | | Off | | s |
| Prod. Filter Shaking Mode | | GPCG | | |
| Temp. Exhaust Air | 30.0 | | 55.0 | ° C. |
| Time | | | | min |

Begin fluidization process. Begin spraying the Surelease 15% solids dispersion until complete using the above noted process parameters. During the initial spraying of the SR coating, the initial product temperature is higher than the glass transition point to get rapid drying. Once sufficient coating of the polymer is applied, the product temperature is decreased to below the glass transition point to avoid agglomeration. The Inlet Air flow is adjusted as necessary.

Connect liquid pumping system to the top coat solution (Opradry Yellow dispersion) and continue spraying until complete. The product temperature is maintained below the glass transition point at steady state, adjusting the inlet air flow rate as necessary.

The pellets are cured, and the batch is screened through # 20 and # 30 mesh screens.

### EXAMPLE 6

### General Process—CPM IR Beads

Using the Chlorpheniramine Maleate layered, seal coated and sifted pellets of Example 4 above, they are charged into a GPCG fluidized bed unit. Sustained Release coat and Top coat are put on the Pellets. The product is cured, and the finished product is screened through #20 and #30 mesh screens.

The process for the IR pellets is identical to that of the SR pellets except for the quantity of Surelease applied, which in the IR pellet is 3% Surelease.

RAW MATERIAL DATA

| | | Quantity per |
|---|---|---|
| Ingredient | %, w/w | Batch (Kg) |
| Purified Water | | 0.26 |
| CPM drug loaded, seal coated | 95.067 | 0.95 |
| Pellets of Example 4 | | |
| Surelease | 2.95 | 0.12 |
| Opadry Yellow | 1.99 | 0.02 |
| Total | 100.00 | 1.08 |

Process produces 4 mg of Chlorpheniramine Maleate per capsule.

Shown below in Table II, is a summary of the processing parameters for the beadlets of the above Examples.

TABLE II
Summary of the Processing Parameters

General range of processing
Conditions ° C.

| | Product | Spray Rates, |
|---|---|---|
| | Temperature | grams/minute |
| Processing Parameters | ° C. | per nozzle |
| PPA pellets | | |
| Drug Loading | 43.9-54.7 | 100-500 |
| Seal Coating | 45.9-58.7 | 100-250 |
| Surelease Coating | 32.0-46.5 | 100-375 |
| Top Coating | 35.5-53.0 | 100-250 |
| CPM Pellets | | |
| Drug Loading | 39.0-52.4 | 100-400 |
| Seal Coating | 35.4-47.5 | 100-200 |
| Surelease Coating | 33.0-46.0 | 100-285 |
| Top Coating | 36.6-45.0 | 150-250 |

### EXAMPLE 7

To the populations of beadlets produced in the above examples, blends of PPA IR, PPA SR, CPM IR, and CPM SR are filled into hard gelatin capsules in ratios of: 1:2:1:1 for the 75/8 form

### EXAMPLE 8

Populations of beadlets of PSE SR release are produced as shown below. The pellets are manufactured in two stages. The first stage consists of making the drug-loaded/seal-coated (DLSC) pellets, whose formula is shown below.

Formula for DLSC Pellets

| Ingredient | %, w/w | mg per Dose |
|---|---|---|
| Pseudoephedrine HCl, USP Powder | 59.057 | 120.000 |
| Non-Pareil 35/40 Mesh Sugar Spheres | 37.354 | 75.901 |
| HPMC E5 Premium, NF (from drug loading) | 1.588 | 3.227 |
| HPMC E5 Premium, NF (from seal-coating) | 2.000 | 4.064 |
| TOTALS | 100.00 | 203.192 |

In a second stage, the SR pellet manufacture, the DLSC pellets are coated with Surelease and then are top coated with Opadry. A “typical formula” of Surelease coated SR pellets is shown below.

Finished pellet formula for a “10%” SR coat level.

10% Surelease* PSE HCl Finished Pellet Formula

| | | Mg per Dose |
|---|---|---|
| | | (120 mg |
| Ingredient | %, w/w | Pseudoephedrine) |
| Pseudoephedrine HCl, USP | 52.080 | 120.000 |
| Powder | | |
| Non-Pareil 35/40 Mesh Sugar | 32.941 | 75.901 |
| Spheres | | |
| HPMC E5 Premium, NF | 3.164 | 7.290 |
| Surelease (25% Solids) | 9.799** | 22.578** |
| Opadry | 2.016 | 4.645 |
| TOTALS | 100.000 | 230.414 |

*% (w/w) at the Surelease-coated beadlet stage.
**Based on dried solids weight.

The pellet formulas at various stages of manufacture of the 10% SR pellets are shown below. The first step in the manufacture of the sustained release pellets is to layer the pseudoephedrine HCl by spraying the drug solution onto the sugar spheres. Hydroxypropylmethylcellulose (HPMC) serves as a binder for the drug (Step 1). The next step involves applying an HPMC “seal” coat to the drug-loaded pellets (Step 2). In the next step (Step 3) the barrier coat, Surelease®, is applied. After the Surelease layer has been applied, a “top-coat”/color-coat layer is applied (Step 4).

| | | |
|---|---|---|
| Ingredient | % (w/w) | mg/120 mg PSE |

Step 1: Drug-Layering Step

| | | |
|---|---|---|
| Sugar Spheres (35/40 Mesh) | 38.12 | 75.90 |
| Pseudoephedrine HCl, | 60.26 | 120.00 |
| USP Powder | | |
| HPMC E5, Premium, NF | 1.620 | 3.23 |
| Totals: | 100.00 | 199.13 |

Step 2: Drug-Loaded/Seal Coated (DLSC) Pellets

| | | |
|---|---|---|
| Drug-Layered Pellets | 98.00 | 199.13 |
| (from step 1) | | |
| HPMC E5, Premium, NF | 2.00 | 4.07 |
| | 100.00 | 203.20 |

Step 3: Functional Coat

| | | |
|---|---|---|
| DLSC Pellets | 90.00 | 203.20 |
| Surelease | 10.00 | 22.58 |
| | 100.00 | 225.78 |

Step 4: Top Coat Application

| | | |
|---|---|---|
| Barrier-Coated Pellets | 97.98 | 225.78 |
| Opadry | 2.02 | 4.65 |
| | 100.00 | 230.43 |

The level of HPMC binder can be varied, with a preferred level in the 1-2% w/w range. The grade of HPMC used (E5), at the 2% level, does not significantly affect drug release through the barrier coat, as it is fairly water-soluble.

Preferably, the functional coat suspension (Surelease®) is applied as a 15% (w/w) Surelease® solids suspension (pseudolatex) in water (Step #3). The “top coat” is applied in Step 4, at the 2.02% (w/w) level and is applied as a 10% (w/w) solids suspension of Opadry in water.

The pellets are then subjected to a “cure” period, of approximately sixty-minutes at a temperature of about 60° C.

In an alternative embodiment of the present example, the functional coat may be applied as a aqueous dispersion having a % (w/w) solids suspension of about 1 to about 60%. Generally, for purposes of commercial manufacture the solids dispersion is diluted to about 25 to 30 w/w. It is more suitably diluted to about 15% w/w for ease of spraying but the diluent may be other than water, such as any soap, surfactant, alchohol, edible oils etc. which will solubilize in the dispersion.

Batches of DLSC pellets were coated with a 25% w/w solids dispersion instead of the 15% w/w dispersion of Step 3 above.

The composition of the solutions/suspensions, mentioned above (drug solution, barrier coat suspension and, top coat suspension), are shown below:

| | |
|---|---|
| Ingredient | % (w/w) |

1. Drug Solution

| | |
|---|---|
| Pseudoephedrine HCl USP Powder | 37.20 |
| Hydroxypropylmethylcellulose E5, USP | 1.00 |
| Purified Water, USP | 61.80 |
| Total: | 100.00 |

2. HPMC Solution

| | |
|---|---|
| Hydroxypropylmethylcellulose | 10.00 |
| Purified Water, USP | 90.00 |
| Total: | 100.00 |

3. Surelease Suspension

| | |
|---|---|
| Surelease (25% solid dispersion) | 60.00 |
| Purified Water, USP | 40.00 |
| Total: | 100.00 |

4. Top Coat Suspension

| | |
|---|---|
| Opadry | 10.00 |
| Purified Water, USP | 90.00 |
| Total: | 100.00 |

### EXAMPLE 9

Using the same process conditions of Example 8 above, sustained release pellets of PSE were made with a 6, 9, 11, 12 and 14% w/w coating level of a functional coat of Surelease.

### EXAMPLE 10

Using process conditions similar to those of Examples 1 to 8 above, immediate release beadlets were produced using Dextromethorphan HBr as the active agent with the following characteristics.

The formulation for producing a beadlet layered with DXM is shown below.

| INGREDIENT | % w/w | mg/dose \hat{ } |
|---|---|---|
| DRUG LAYERED BEADLETS | | |
| Dextromethorphan HBr | 47.170 | 30.000 |
| Sugar Spheres NF | 47.170 | 30.000 |
| Hydroxypropylmethylcellulose, 5 cps | 5.660 | 3.600 |
| Water, Purified, USP. | — | 137.400 |
| | 100.000 | 63.600 |

Notes:
\hat{ }Dose denotes 30 mg of Dextromethorphan HBr per capsule

Using the drug layered beadlets of Step A above, immediate release beadlets of DXM are produced coated with an HPC seal coat in accordance with the following formulation.

DXM IMMEDIATE RELEASE BEADLETS

| | | |
|---|---|---|
| DRUG LAYERED BEADLETS | 98.000 | 63.600 |
| Hydroxypropylmethylcellulose, 5 cps | 2.000 | 1.298 |
| Water, Purified, USP. | — | 11.682 |
| | 100.000 | 64.898 |

Notes:
“*” Denotes water evaporated during processing

Using similar Drug Layering Processing Parameters to the CPM, PSE, and PPA examples above, a GPCG-15, 12″ Wurster column is used to apply both the Drug Layer and the Seal Coat.

While unit specific for manufacturing conditons, it is suitable to apply the DXM suspension at a spray rate of 100 to 400 grams per minute/per nozzle; the product temperature is kept within a temperature range of about 43+/−3° C. Because the drug is sprayed as a suspension the temperature may be increased up to 50° C. The lower end of the temperature range is down to about 32° C. depending upon unit size. Therefore the range is from about 32 to about 50° C.

### EXAMPLE 11

After the drug loaded sealed coated beadlets of Example 9 are prepared, the pellets are now coated with the release rate agent, with set up and process parameters for two formulas, the 5 and 7% SR formulations are shown below.

For a 5% sustained release formulation:

5% SR Formulation

| | | |
|---|---|---|
| INGREDIENT | % w/w | mg/dose \hat{ } |

SURELEASE COAT

| | | |
|---|---|---|
| Dextromethorphan DLSC Pellets from | 95.00 | 64.898 |
| the example above | | |
| Surelease (25% solids Dispersion) | 5.00 | 3.416 |
| Water, Purified, USP. | — | 9.108 |
| total | 100.00 | 68.314 |

DXM IMMEDIATE RELEASE BEADLET

| | | |
|---|---|---|
| Surelease Coated Pellets | 98.00 | 68.314 |
| Opadry White | 2.00 | 1.394 |
| Water, Purified, USP. | — | |
| total | 100.00 | 69.708 |

Notes:
\hat{ }Dose denotes 30 mg of Dextromethorphan HBr per capsule

For a 7% SR Formulation

| | | |
|---|---|---|
| INGREDIENT | % w/w | mg/dose \hat{ } |

SURELEASE COAT

| | | |
|---|---|---|
| Dextromethorphan DLSC Pellets | 93.00 | 64.898 |
| Surelease (25% solids Dispersion) | 7.00 | 4.885 |
| Water, Purified, USP. | — | 13.027 |
| total | 100.00 | 69.783 |

DXM IMMEDIATE RELEASE BEADLET

| | | |
|---|---|---|
| Surelease Coated Pellets | 98.00 | 69.783 |
| Opadry White | 2.00 | 1.424 |
| Water, Purified, USP. | — | |
| total | 100.00 | 71.207 |

Notes:
\hat{ }Dose denotes 30 mg of Dextromethorphan HBr per capsule

Using process conditions similar to those above in Examples 1 to 9, the functional coat and top coat films were applied, and cured.

### EXAMPLE 12

Similar to Example 11 above, sustained release pellets with a 9% w/w functional coat layer were also made.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1-23. (canceled)

24. An aqueous coating process for the manufacture of sustained release beadlets of a water soluble active agent coated with a water swellable polymer as the sustained releasing agent which process comprises
a) applying a seal coat of a protective polymer to a micronized dextromethorphan drug loaded sphere;
b) applying a coating of an aqueous water swellable polymeric dispersion to the sphere of step a); wherein the aqueous water swellable polymeric dispersion of step b) is a pseudolatex ethyl cellulose dispersion having a glass transition point of about 38 to 41° C.; and which process for applying said dispersion utilizes atmospheric conditions exhibiting a dew point of 9+/−5° C.

25. The process according to claim 24 wherein the dew point is 9+/−3° C.

26. The process according to claim 24 wherein the drug loaded sphere is a sugar sphere or microcrystalline cellulose sphere coated with a water soluble active agent.

27. The process according to claim 24 wherein the drug loaded sphere is a spherionized pellet.

28. (canceled)

29. The process according to claim 24 wherein the pseudolatex ethyl cellulose dispersion is Surelease®.

30-34. (canceled)

35. The process according to claim 24 wherein the temperature of the product is lowered to below the glass transition point of the polymeric dispersion and maintained at a steady state temperature after a sufficient amount of the water swellable polymeric dispersion has been applied.

36. (canceled)

37. (canceled)

38. The process according to claim 24 wherein the seal coat is hydroxypropylmethylcellulose.

39. The process according to claim 24 wherein the seal coat is polyvinyl alcohol.

40. The process according to claim 38 wherein the seal coat applied is from about 1 to 7% weight gain.

41-61. (canceled)

62. A pharmaceutical product which comprises a sustained release (SR) phase of micronized dextromethorphan (DXM) loaded onto an inert sphere, wherein the sphere is coated with about 0.5 to about 15% (weight gain) of a pseudolatex water swellable polymer dispersion.

63. A product according to claim 62 wherein the dextromethorphan spheres are coated with about 3 to about 10% (weight gain) of a pseudolatex water swellable polymer dispersion.

64. A product according to claim 62 wherein the dextromethorphan spheres are coated with about 4 to 7% (weight gain) of the pseudolatex water swellable polymer dispersion.

65. A product according to claim 67 comprising a drug content of DXM between 30 to 70% w/w of dextromethorphan in the sustained release phase.

66. A product according to claim 65 comprising a drug content load of DXM between 40 to 60% w/w of dextromethorphan in the sustained release phase.

67. A product according to claim 62 further comprising an immediate release phase of dextromethorphan.

68. An immediate release (IR) or sustained release (SR) product according to claim 67 having an AUC, Cₘₐₓ, and tₘₐₓ, according to FIG. 20.

69. An immediate release (IR) or sustained release (SR) product according to claim 67 having an AUC, Cₘₐₓ, and tₘₐₓ, according to FIG. 21.

70. An immediate release (IR) or sustained release (SR) product according to claim 67 having an AUC, Cₘₐₓ, and tₘₐₓ, according to FIG. 22.

71. A product according to claim 67 wherein the weight ratio of immediate release DXM to sustained release DXM is 0:100 to 100:0.

72. A product according to claim 67 wherein the weight ratio of immediate release DXM to sustained release DXM is 1:1.

73. A product according to claim 72 which contains 30 mg of immediate release (IR) DXM:30 mg sustained release (SR) DXM; or 2.5 mg immediate release (IR) and 2.5 mg sustained release (SR) DXM.

74-76. (canceled)

77. The product according to claim 70 admixed with 200 to 1200 mg ibuprofen.

78. A product according to claim 62 wherein the pseudolatex ethyl cellulose is a dispersion containing a plasticizer.

79. A product according to claim 24 wherein the pseudolatex ethyl cellulose dispersion of ethylcellulose is Surelease®.

80. (canceled)

81. The product according to claim 62 wherein the micronized Dextromethorphan HBr has a particle size of 25 microns or less.

82. The product according to claim 81 wherein the micronized DXM HBr has a particle size of 10 microns or less.

83. The product according to claim 81 wherein at least 90% of the particles are 5 microns or less.

84. A pharmaseutical product comprising a immediate release (IR) formulation of micronized dextromethorphan and particle size of the dextromethorphan is 25 microns or less.

85. (canceled)

86. The product according to claim 84 wherein the micronized DXM HBr has a particle size of 10 microns or less.

87. The product according to claim 86 wherein at least 90% of the particles are 5 microns or less.

88. Micronized Dextromethorphan HBr having a particle size of less than 50 microns.

89. The micronized DXM according to claim 88 having a particle size of less than 25 microns.

90. The micronized DXM according to claim 89 having a particle size of less than 10 microns.

91. The micronized DXM according to claim 88 wherein at least 90% of the particles are 5 microns or less.

92. The micronized DXM particles of claim 88 produced by air-jet milling, grinding or impact milling.

93. The process according to claim 27 wherein the amount of dextromethorphan on the spherionized pellet is about 30 to 70% w/w drug load.

94. The process according to claim 93 wherein the amount of ethyl cellulose dispersion applied to the spherionized pellet of dextromethorphan is from about 0.5 to 15%.

95. The process according to claim 24 wherein the sphere of part a) containing dextromethorphan as the active agent is coated with the aqueous water swellable polymeric dispersion initially at a product temperature above the glass transition point of the polymeric dispersion.

96. The process according to claim 95 wherein the temperature of the product is lowered to below the glass transition point of the polymeric dispersion and maintained at a steady state temperature after a sufficient amount of the water swellable polymeric dispersion has been applied.

97. The process according to claim 80 wherein the seal coat is hydroxypropylmethylcellulose.

98. The process according to claim 80 wherein the seal coat is polyvinyl alcohol.

99. The process according to claim 97 or wherein the seal coat applied is from about 1 to 7% weight gain.

100. The process according to claim 80 wherein the sustained release coated beadlet is cured for about 1 hour at a temperature of about 60° C.

101. A product produced by the process according to claim 94.

102. (canceled)

103. (canceled)

104. The product according to claim 78 wherein the plasticizer is a medium chain triglyceride.

105. The product according to claim 104 wherein the medium chain triglyceride is coconut oil.
